(19) United States
(12) Patent Application Publication
(10) Pub. No.: US 2024/0415800 A1
(43) Pub. Date: Dec. 19, 2024

(54) DOPA AND CAFFEIC ACID ANALOGS AS NOVEL GCPII INHIBITORS

(71) Applicant: The Johns Hopkins University, Baltimore, MD (US)

(72) Inventors: Rana RAIS, West Friendship, MD (US); Barbara SLUSHER, Kingsville, MD (US); Takashi TSUKAMOTO, Ellicott City, MD (US)

(21) Appl. No.: 18/695,418

(22) PCT Filed: Oct. 11, 2022
(86) PCT No.: PCT/US2022/077925

§ 371 Date: Mar. 26, 2024

Related U.S. Application Data
(60) Provisional application No. 63/254,344, filed on Oct. 11, 2021.

(51) Int. Cl. A61K 31/265; A61K 31/165; A61K 31/167; A61K 31/192; A61K 31/198; A61K 31/222; A61K 31/24; A61K 31/496; A61K 31/50; A61K 31/519; C07C 69/732; C07C 69/96; C07C 235/34; C07C 235/36; C07C 235/38; C07C 237/22

(52) U.S. Cl. CPC A61K 31/265; A61K 31/165; A61K 31/167; A61K 31/192; A61K 31/198; A61K 31/222; A61K 31/24; A61K 31/496; A61K 31/50; A61K 31/519; C07C 69/732; C07C 69/96; C07C 235/34; C07C 235/36; C07C 235/38; C07C 237/22; C07B 2200/05; C07C 2601/02

(57) ABSTRACT

Caffeic Acid, L-DOPA, and D-DOPA and prodrugs thereof for treating a disease, condition, or disorder associated with excess glutamate carboxypeptidase II (GCP-II) are disclosed.

### FEDERALLY SPONSORED RESEARCH OR DEVELOPMENT

This invention was made with government support under grant AG068130 awarded by the National Institutes of Health. The government has certain rights in the invention.

### BACKGROUND

Glutamate carboxypeptidase II (GCPII), also known as N-acetyl-L-aspartyl-L-glutamate peptidase I (NAALADase I), NAAG peptidase, or prostate-specific membrane antigen (PSMA), is a class II membrane glycoprotein, which catalyzes the hydrolysis of N-acetylaspartylglutamate (NAAG) to glutamate and N-acetylaspartate (NAA). NAAG is one of the most abundant neuropeptides in the mammalian brain and regulates glutamate neurotransmission through its agonist activity at the metabotropic glutamate receptor 3 (mGluR3), as well as its ability to release glutamate, which can act at a variety of glutamate receptors.

The utility of GCPII inhibitors has been demonstrated in several animal models of nervous system disease where excess glutamate transmission is presumed pathogenic and/or activation of the mGluR3-TGFβ pathway is neuroprotective. These models include peripheral neuropathy, inflammatory and neuropathic pain, brain and spinal cord ischemia, traumatic brain injury, amyotrophic lateral sclerosis, epilepsy, drug abuse, cognitive impairment in multiple sclerosis, and schizophrenia. Although many GCPII inhibitors have shown promise in preclinical studies, targeted delivery and blood brain barrier penetration remain major obstacles in clinical candidate development.

### SUMMARY

In some aspects, the presently disclosed subject matter provides a compound of formula (I):

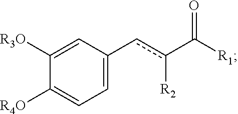

wherein:
   [symbol] indicates that the bond can be a single or a double bond;
   R₁ is:
      —OR₅, wherein R₅ is H or C₁-C₈ alkyl; or
      —NR₇R₈, wherein R₇ is H or C₁-C₄ alkyl and R₈ is selected from the group consisting of C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
   R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or
      —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
   R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8;
   provided that if R₁ is —OR₅, then R₃, R₄, and R₅ cannot all be H, and that if R₃ and R₄ are each H, then R₇ and R₈ cannot both be methyl; and
   stereoisomers and pharmaceutically acceptable salts thereof.

In certain aspects, R₁ is —OR₅, and R₅ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In certain aspects, R₁ is —NR₇R₈, and R₇ is H or C₁-C₄ alkyl and R₈ is selected from the group consisting of C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

In certain aspects, R₂ is —NR₁₁R₁₂, wherein Ru is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8.

In certain aspects, R₃ and R₄ are each H, provided that if R₁ is —OR₅, then R₅ cannot be H.

In certain aspects, R₃ and R₄ are each independently selected from the group consisting of —C(═O)—CH₃, —C(═O)—C(CH₃)₃, and —CH₂—O—C(═O)—O—CH(CH₃)₂.

In particular aspects, the compound of formula (I) is selected from the group consisting of:

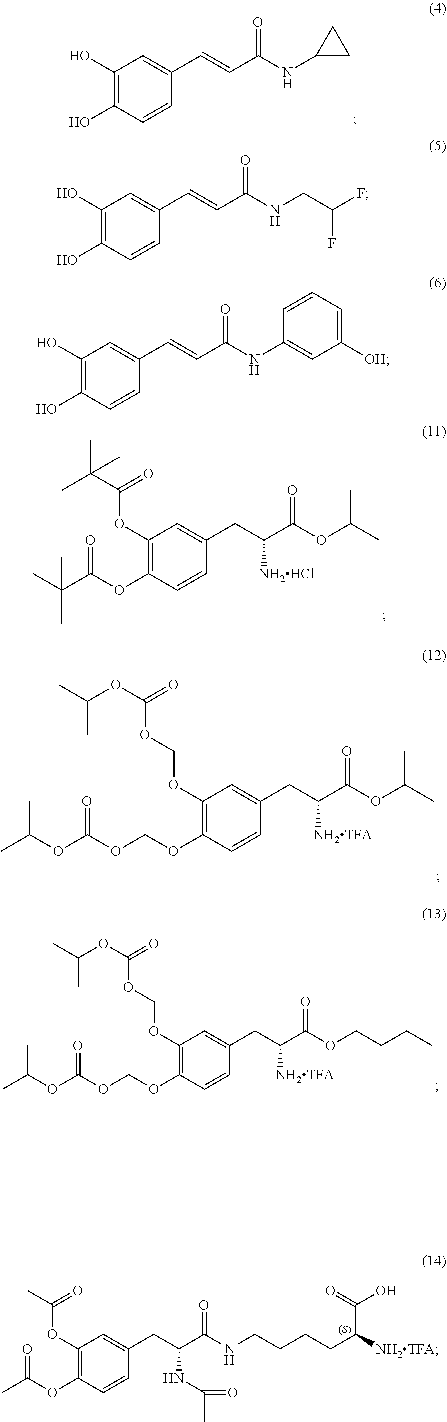

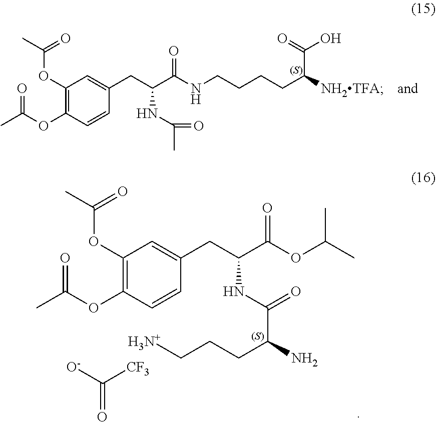

In some aspects, the presently disclosed subject matter provides a pharmaceutical composition comprising a compound of any one of claims 1-8 and a pharmaceutically acceptable carrier, diluent, or excipient.

In some aspects, the presently disclosed subject matter provides a method for treating a disease, disorder, or condition, the method comprising administering to a subject in need of treatment thereof, a therapeutically effective amount of L-DOPA, caffeic acid, D-DOPA, or a compound of formula (I):

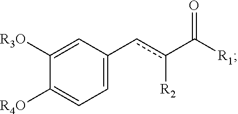

wherein:
   [symbol] indicates that the bond can be a single or a double bond;
   R₁ is:
      —OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
      —NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
   R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or
      —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
stereoisomers and pharmaceutically acceptable salts thereof.

In certain aspects, R₁ is —OR₅, and R₅ is selected from the group consisting of H, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In certain aspects, R₁ is —OR₅, and R₅ is H or —O—(CH₂)ₙ—R₆, wherein R₆ is substituted or unsubstituted phenyl.

In certain aspects, R₁ is —NR₇R₈, and R₇ is H or C₁-C₄ alkyl and R₈ is selected from the group consisting of H, C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

In certain aspects, R₂ is —NR₁₁R₁₂, wherein R₁ is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8.

In certain aspects, R₃ and R₄ are each H.

In certain aspects, R₃ and R₄ are each independently selected from the group consisting of —C(═O)—CH₃, —C(═O)—C(CH₃)₃, and —CH₂—O—C(═O)—O—CH(CH₃)₂.

In certain aspects, the compound of formula (I) is selected from the group consisting of:

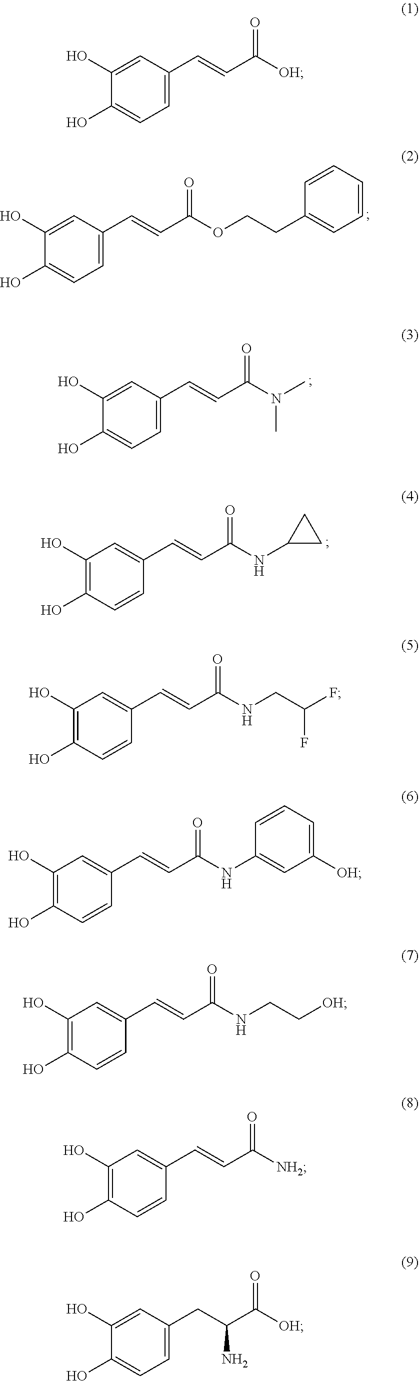

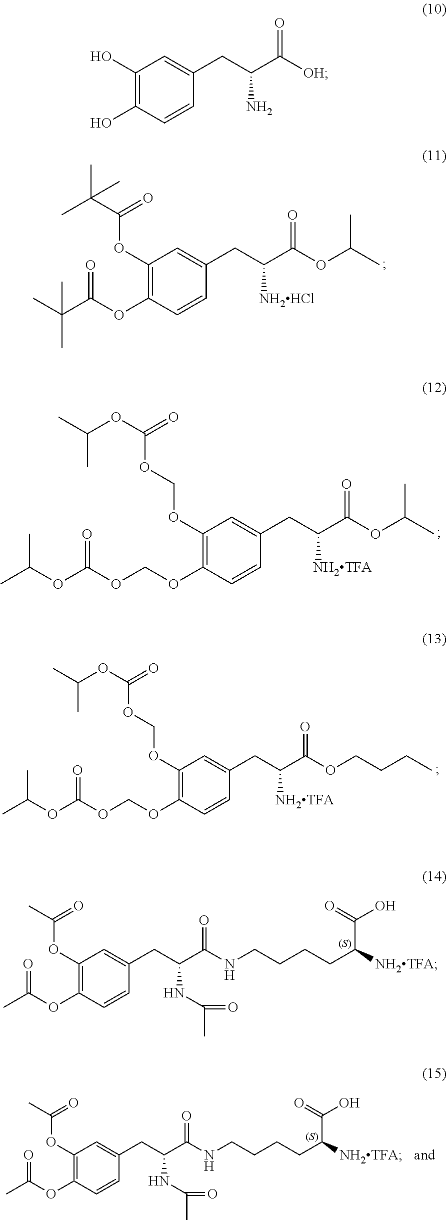

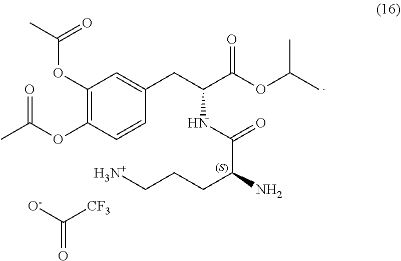

In some aspects, the disease, disorder, or condition is a central nervous system (CNS) or an inflammatory disease, disorder, or condition.

In certain aspects, the disease, disorder, or condition is selected from the group consisting of ischemic stroke, traumatic brain injury, traumatic spinal cord injury, amyotrophic lateral sclerosis (ALS), Parkinson's disease, Huntington's disease, epilepsy, neuropathic pain, inflammatory pain, chemotherapy-induced neuropathy, diabetic neuropathy, schizophrenia, multiple sclerosis, cognition impairment, brain cancer, HIV-associated neurocognitive disorder, cognition impairment associated with neurodegenerative or neuropsychiatric conditions, inflammatory bowel disease (IBD), and neurological conditions as a result of drug abuse or drug addiction.

In particular aspects, the method comprises intranasally administering a therapeutically effective amount of a compound of formula (I).

In certain aspects, the presently disclosed method further comprises administering L-DOPA, caffeic acid, D-DOPA, or a compound of formula (I) in combination with a D-amino acid oxidase (DAAO) inhibitor. In certain aspects, the DAAO inhibitor is selected from sodium benzoate, risperidone, blonanserin, and luvadaxistate (TAK-831). In particular aspects, the DAAO inhibitor comprises sodium benzoate.

In certain aspects, the method further comprises administering L-DOPA in combination with carbidopa.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a scheme showing a derivatization reaction for D-DOPA, L-DOPA, and L-DOPA-ring-d₃ with acetic anhydride in presence of 0.2 M sodium bicarbonate, pH 8.3. Three acetyl groups were incorporated (i.e., two on the catecholic hydroxyls and one on primary amine) forming a nonpolar compound that could be retained on a reverse phase column;

FIG. 2A shows representative LC chromatograms of acetylated D-DOPA (top), and L-DOPA-d₃ (bottom)—inserts show respective MS signals resulting from PRM transitions (D-DOPA: 322.0927→238.0718, L-DOPA-dⱼ 325.1115→240.0844) observed in the negative mode:

FIG. 2B is a standard calibration curve of derivatized D-DOPA in mouse plasma (R=0.9998. Range=10 nmol/mL-100 nmol/mL, LLOQ=30 nmol/mL);

FIG. 2C is the internal standard precision in mouse plasma (% RSD=7 3%);

FIG. 2D is a standard calibration curve of derivatized D-DOPA in mouse brain homogenate (R²=0.9994, Range=10 nmol/g-3 nmol/g, LLOQ=30 nmol/g);

FIG. 2E is the internal standard precision in mouse brain homogenate (% RSD=20.8%);

FIG. 3A and FIG. 3B demonstrate the pharmacokinetics of D-DOPA in mouse plasma and brain. (FIG. 3A) Time-dependent PK plot (±SEM) of D-DOPA dosed intravenously (IV) (10 mg/kg) and orally (PO) (30 mg/kg) (see also FIG. 9a); and (FIG. 3B) Time-dependent PK plot (±SEM) of D-DOPA dosed intranasally (IN) (3.4 mg/kg) and orally (PO) (30 mg/kg) in CD1 mice:

FIG. 4 shows the GCPII activity in cortex after PO (30 mg/kg) dose of D-DOPA; GCPII inhibition was found to be significant at 1 h post oral dose;

FIG. 5 shows the pharmacokinetics of D-DOPA and representative prodrugs 1-5, and 7 in rat plasma at 30 min ater dosing (IN_{surg}) @ 1 mpk equiv. to D-DOPA, demonstrating high plasma exposure for representative ester-linked prodrugs,

FIG. 6 shows the pharmacokinetics of D-DOPA and representative prodrugs 1-5, and 7 in rat brain at 30 min after dosing (IN_{surg}) @ 1 mpk equiv. to D-DOPA, demonstrating low exposure in brain at 30 min for representative ester-linked prodrugs;

FIG. 7 shows the % brain penetration of D-DOPA for INs_{surg} dosing of representative prodrugs 1-5, and 7, for which slow conversion of prodrugs with amide linkages is observed;

FIG. 8 shows the chemical structures of representative prodrugs tested in FIG. 5, FIG. 6, and FIG. 7;

FIG. 9a, FIG. 9b, FIG. 9c, FIG. 9d, and FIG. 9e show time-dependent PK profile of D-DOPA in CD1 mice. (FIG. 9a) PK profile following IV dose of 10 mg/kg. (FIG. 9b) PK profile following monotherapy with D-DOPA (PO—50 mg/kg). (FIG. 9c) PK profile following combination therapy with D-DOPA (PO—50 mg/kg) and sodium benzoate (IP—400 mg/kg). (FIG. 9d) GCPII activity in CD1 mouse brain at 30 min when dosed orally with D-DOPA at 50 mg/kg with or without a DAAO inhibitor, sodium benzoate (400 mg/kg IP). Statistical analysis was performed used two-tailed, two-population t-test; ***p<0.001, **p 0.01; *p<0.05. (FIG. 9e) PK parameters of D-DOPA in CD1 mouse plasma and brain following IV dose of 10 mg/kg, or PO dose of D-DOPA at 50 mg/kg with or without 400 mg/kg IP dose of sodium benzoate. Calculated using noncompartmental analysis in WinNonlin, with data expressed as mean or mean±SEM (n=3 mice/time point); and

FIG. 10a, FIG. 10b, and FIG. 10c illustrate the mechanism of inhibition of GCPII by D-DOPA. (FIG. 10a) Rate of reaction plotted against substrate (NAAG) concentration, in the presence of several concentrations of D-DOPA. Recombinant human GCPII enzyme (40 pM) was incubated with increasing concentrations of radiolabeled NAAG and coupling reagents for 2 h at 37° C. (FIG. 10b) Lineweaver-Burk plot to illustrate the non-competitive inhibition by D-DOPA. (FIG. 10c) Secondary plot (K_{M} app/Vₘₐₓ vs. [D-DOPA]) to obtain the binding constant (Kᵢ=−X intercept) of D-DOPA. (d) Vₘₐₓ and K_{M} values were obtained from non-linear regression fits to Michaelis-Menten kinetics using GraphPad Prism.

Having thus described the presently disclosed subject matter in general terms, reference will now be made to the accompanying Drawings, which are not necessarily drawn to scale, and wherein:

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Prodrugs of Caffeic Acid, L-DOPA, and D-DOPA

In some embodiments, the presently disclosed subject matter provides the identification of L-DOPA, D-DOPA and caffeic acid as inhibitors of GCPII. Using these inhibitors as scaffolds, the presently disclosed subject matter provides prodrugs of L-DOPA, D-DOPA, and caffeic acid as compounds of formula (I):

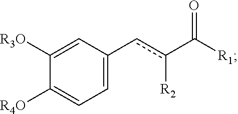

wherein:
   [symbol] indicates that the bond can be a single or a double bond;
   R₁ is:
      —OR₅, wherein R₅ is selected from the group consisting of H, C₁-C₈ alkyl; or
      —NR₇R₈, wherein R₇ is H or C₁-C₄ alkyl and R₈ is selected from the group consisting of C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
   R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or
      —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
   R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8;
   provided that if R₁ is —OR₅, then R₃, R₄, and R₅ cannot all be H, and that if R₃ and R₄ are each H, then R₇ and R₈ cannot both be methyl; and stereoisomers and pharmaceutically acceptable salts thereof.

In certain embodiments, R₁ is —OR₅, and R₅ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In certain embodiments, R₁ is —NR₇R₈, and R₇ is H or C₁-C₄ alkyl and R₈ is selected from the group consisting of C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

In certain embodiments, R₂ is —NR₁₁R₁₂, wherein R₁₁ is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8.

In certain embodiments, R₃ and R₄ are each H, provided that if R₁ is —OR₅, then R₅ cannot be H.

In certain embodiments, R₃ and R₄ are each independently selected from the group consisting of —C(═O)—CH₃, —C(═O)—C(CH₃)₃, and —CH₂—O—C(═O)—O—CH(CH₃)₂.

In particular embodiments, the compound of formula (I) is selected from the group consisting of:

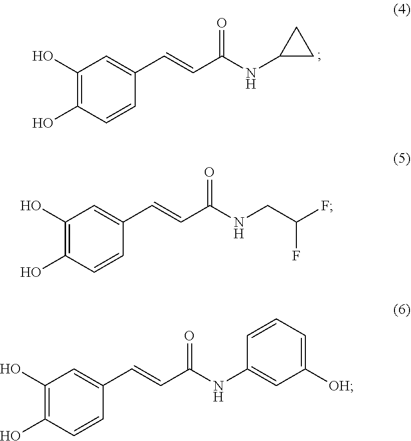 [SMILES: O=C(/C=C/c1ccc(O)c(O)c1)NC1CC1 ; ";" ; O=C(/C=C/c1ccc(O)c(O)c1)NCC(F)F ; O=C(/C=C/c1ccc(O)c(O)c1)Nc1cccc(O)c1]

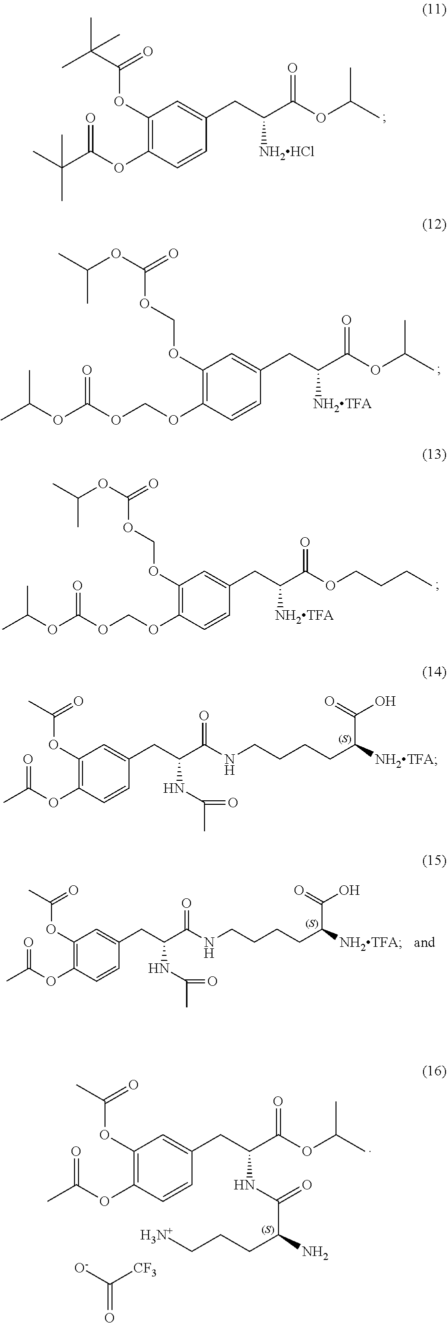

More particularly, as provides in Table 1, the following prodrugs of these GCPII inhibitors have been synthesized prodrugs to enhance permeability and to augment brain penetration.

TABLE 1
Catechol Scaffolds for GCPII Inhibition

| | | | |
|---|---|---|---|
| # | Compound | Structures | IC₅₀ |

Caffeic Acid Scaffold and its Prodrugs

| | | | |
|---|---|---|---|
| 1 | Caffeic acid | 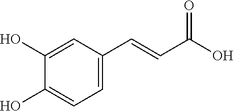 [SMILES: O=C(O)/C=C/c1ccc(O)c(O)c1] | 100 nM |
| 2 | CAPE | 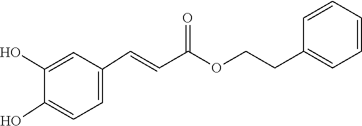 [SMILES: O=C(/C=C/c1ccc(O)c(O)c1)OCCc1ccccc1] | — |
| 3 | JHU-5144 | 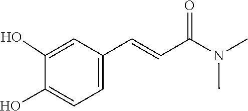 | — |
| 4 | JHU-5145 | 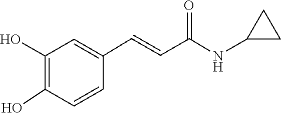 [SMILES: O=C(/C=C/c1ccc(O)c(O)c1)NC1CC1] | — |
| 5 | JHU-5146 | 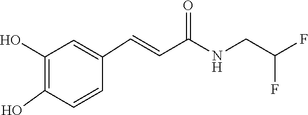 [SMILES: O=C(/C=C/c1ccc(O)c(O)c1)NCC(F)F] | — |
| 6 | JHU-5147 | 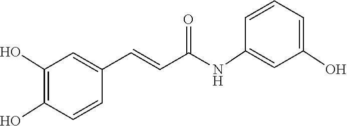 [SMILES: O=C(/C=C/c1ccc(O)c(O)c1)Nc1cccc(O)c1] | — |
| 7 | TBD | 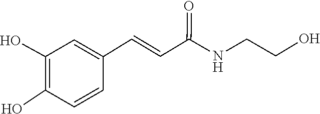 [SMILES: O=C(/C=C/c1ccc(O)c(O)c1)NCCO] | — |
| 8 | TBD | 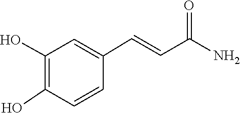 [SMILES: NC(=O)/C=C/c1ccc(O)c(O)c1] | — |

L-DOPA Scaffold and its Prodrugs

| | | | |
|---|---|---|---|
| 9 | L-DOPA | 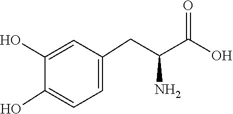 [SMILES: N[C@@H](Cc1ccc(O)c(O)c1)C(=O)O] | 400 nM |

D-DOPA Scaffold and its Prodrugs

| | | | |
|---|---|---|---|
| 10 | D-DOPA | 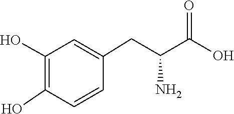 [SMILES: N[C@H](Cc1ccc(O)c(O)c1)C(=O)O] | 200 nM |
| 11 | Prodrug-1 | 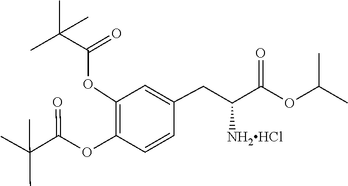 | — |
| 12 | Prodrug-2 | 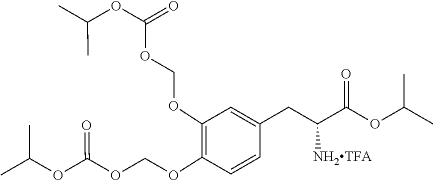 | — |
| 13 | Prodrug-3 | 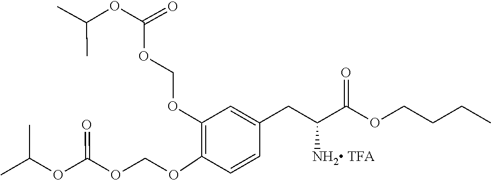 | — |
| 14 | Prodrug-4 | 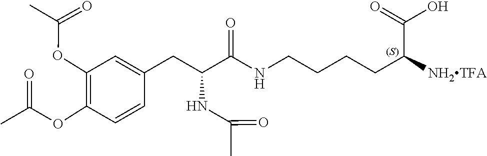 | — |
| 15 | Prodrug-5 | 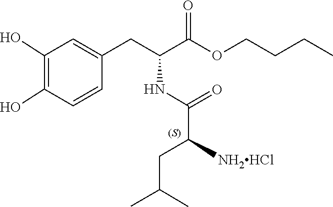 | — |
| 16 | Prodrug-7 | 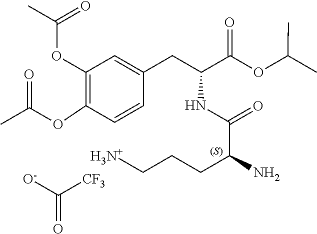 | — |

### B. Methods for Treating a Disease, Disorder, or Condition Associated with Excess Glutamate Carboxypeptidase II (GCPII)

In some embodiments, the presently disclosed subject matter provides methods for treating a disease, disorder, or condition associated with excess glutamate carboxypeptidase II (GCPII).

Glutamate carboxypeptidase II (GCPII) is a 94 kD class II membrane bound zinc metalloenzyme which catalyzes the hydrolysis of the abundant neuropeptide N-acetylaspartylglutamate (NAAG) to glutamate. GCPII is a well-established therapeutic target in neurological diseases wherein excess glutamate is presumed pathogenic.

As used herein, the term “excess,” as in “an excess of GCPII,” refers to a level of GCPII in a subject having or suspected of having a disease, disorder, or condition associated with GCPII compared to a level of GCPII in a normal subject, i.e., a subject who does not have or is not suspected of having a disease, disorder, or condition associated with excess GCPII, such as an increase of approximately 50%, 100%, 200%, 300%, 400%, 500%, or more.

In some embodiments, performing the presently disclosed method results in inhibiting excess GCP-II activity in a subject. In other embodiments, performing the presently disclosed method results in almost 100% inhibition of GCP-II enzyme activity in the olfactory bulb and cortex of the brain and at least 70% inhibition in the cerebellum of the brain. As used herein, the term “inhibit” means to decrease or diminish the excess GCP-II activity found in a subject. The term “inhibit” also may mean to decrease, suppress, attenuate, diminish, arrest, or stabilize the development or progression of a disease, disorder, or condition. Inhibition may occur, for e.g., by at least 10%, 20%, 30%, 40%, 50%, 60%, 70%, 80%, 90%, 95%, 98%, 99%, or even 100% compared to an untreated control subject or a subject without the disease or disorder.

As used herein, an “inhibitor” of GCP-II is a molecule that generally inhibits or decreases the activity of GCP-II. In some embodiments, small molecule GCP-II inhibitors, directly or indirectly, increase extracellular NAAG and decrease extracellular glutamate.

The inhibitor may interact with GCP-II directly or may interact with another molecule that results in a decrease in the activity of GCP-II.

Accordingly, in some embodiments, the presently disclosed subject matter provides a method for treating a disease, disorder, or condition, the method comprising administering to a subject in need of treatment thereof, a therapeutically effective amount of L-DOPA, caffeic acid, D-DOPA, or a compound of formula (I):

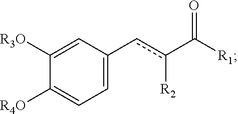

wherein:
   [symbol] indicates that the bond can be a single or a double bond;
   R₁ is
   —OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
      —NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
   R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or
      —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
   R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
   stereoisomers and pharmaceutically acceptable salts thereof.

In certain embodiments, R₁ is —OR₅, and R₅ is selected from the group consisting of H, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

In certain embodiments, R₁ is —OR₅, and R₅ is H or —O—(CH₂)ₙ—R₆, wherein R₆ is substituted or unsubstituted phenyl.

In certain embodiments, R₁ is —NR₇R₈, and R₇ is H or C₁-C₄ alkyl and R₈ is selected from the group consisting of H, C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

In certain embodiments, R₂ is —NR₁₁R₁₂, wherein R₁₁ is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8.

In certain embodiments, R₃ and R₄ are each H.

In certain embodiments, R₃ and R₄ are each independently selected from the group consisting of —C(═O)—CH₃, —C(═O)—C(CH₃)₃, and —CH₂—O—C(═O)—O—CH(CH₃)₂.

In certain embodiments, the compound of formula (I) is selected from the group consisting of:

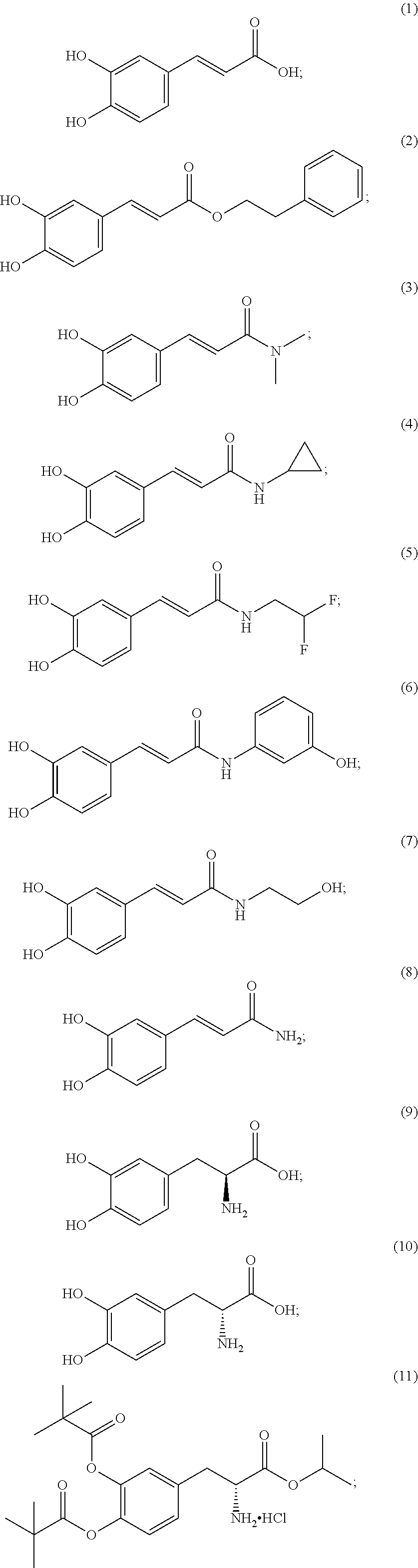

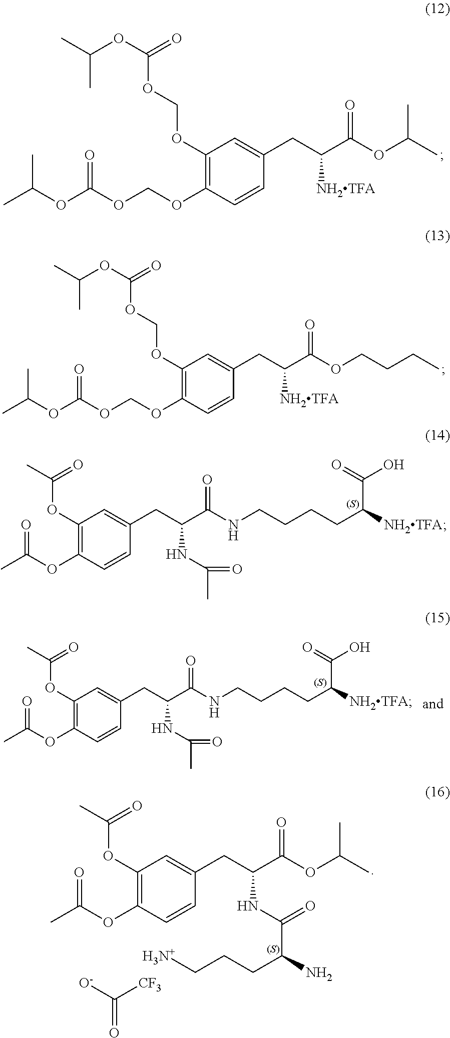

In some embodiments, the method comprises administering a therapeutically effective amount of L-DOPA.

In some embodiments, the method comprises administering a therapeutically effective amount of caffeic acid.

In some embodiments, the method comprises administering a therapeutically effective amount of D-DOPA.

In some embodiments, the method comprises administering a therapeutically effective amount of compound 11.

In some embodiments, the method comprises administering a therapeutically effective amount of compound 12.

In some embodiments, the method comprises administering a therapeutically effective amount of compound 13.

In some embodiments, the method comprises administering a therapeutically effective amount of compound 14.

In some embodiments, the method comprises administering a therapeutically effective amount of compound 15.

In some embodiments, the method comprises administering a therapeutically effective amount of compound 16.

In some embodiments, the disease, disorder, or condition is associated with an abnormal level of glutamate carboxypeptidase II (GCPII).

In some embodiments, the disease, disorder, or condition is associated with excess GCPII.

In some embodiments, the disease, disorder, or condition is associated with abnormal glutamatergic transmission.

In some embodiments, the disease, disorder, or condition is a central nervous system (CNS) or an inflammatory disease, disorder, or condition.

In certain embodiments, the disease, disorder, or condition is selected from the group consisting of ischemic stroke, traumatic brain injury, traumatic spinal cord injury, amyotrophic lateral sclerosis (ALS), Parkinson's disease, Huntington's disease, epilepsy, neuropathic pain, inflammatory pain, chemotherapy-induced neuropathy, diabetic neuropathy, schizophrenia, multiple sclerosis, cognition impairment, brain cancer, HIV-associated neurocognitive disorder, cognition impairment associated with neurodegenerative or neuropsychiatric conditions, inflammatory bowel disease (IBD), and neurological conditions as a result of drug abuse or drug addiction.

In particular embodiments, the compound of formula (I) is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. It has been found that the presently disclosed small molecule GCP-II inhibitors are much more effective at penetrating the brain and peripheral nervous system when administered intranasally.

As used herein, the term “peripheral nervous system” includes the part of the nervous system comprising the nerves and ganglia on the outside of the brain and spinal cord. The peripheral nervous system connects the central nervous system to the limbs and organs and acts as a communication relay between the brain and the extremities. The presently disclosed small molecule GCP-II inhibitors can access the peripheral nervous system through the blood.

Intranasal administration generally allows the active agent to bypass first pass metabolism, thereby enhancing the bioavailablity of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the brain and/or peripheral nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol mnonocaprate (Capmul™), mixtures of mono-, di-, and triglycerides and mono- and di-fatty esters of PEG (Labrafil™), palmitate, glycerol monostearate, and phospholipids can be used to administer the presently disclosed GCP-II inhibitors intranasally.

The presently disclosed GCP-II inhibitors also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed GCP-II inhibitors include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyaciylates, such as poly(acrylic acid) (e.g., Carbopol® 974P), functionalized mucoadhesive polymers, such as polycarbophil, hyaluronan, and amberlite resin, and chitosan (2-armino-2-deoxv-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug. Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.).

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

In certain embodiments, the presently disclosed method further comprises administering L-DOPA, caffeic acid, D-DOPA, or a compound of formula (I) in combination with a D-amino acid oxidase (DAAO) inhibitor. In particular embodiments, the DAAO inhibitor comprises sodium benzoate. Other DAAO inhibitors include, but are not limited to, risperidone, blonanserin, luvadaxistate (TAK-831) and those compounds disclosed in Molla G. Competitive Inhibitors Unveil Structure/Function Relationships in Human D-Amino Acid Oxidase. Front Mol Biosci. 2017 Nov. 27; 4:80; Smith S M, Uslaner J M, Hutson P H. The Therapeutic Potential of D-Amino Acid Oxidase (DAAO) Inhibitors. Open Med Chem J. 2010 May 27; 4:3-9; and Ferraris D V, Tsukamoto T. Recent advances in the discovery of D-amino acid oxidase inhibitors and their therapeutic utility in schizophrenia. Curr Pharm Des. 2011; 17(2).103-11, each of which is incorporated herein by reference in its entirety. Other novel DAAO inhibitors are disclosed in International PCT Patent Application Publication No. WO2014025993 for Inhibitors of D-Amino Acid Oxidase, to Tsukamoto et al., published Feb. 13, 2014, which is incorporated herein by reference in its entirety.

In certain embodiments, the method further comprises administering L-DOPA in combination with carbidopa.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
   Q_{A} is the concentration of a component A, acting alone, which produced an end point in relation to component A;
   Qₐ is the concentration of component A, in a mixture, which produced an end point;
   Q_{B} is the concentration of a component B, acting alone, which produced an end point in relation to component B; and
   Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

In another aspect, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

As used herein the terms “bicycloalkyl” and “bicycloheteroalkyl” refer to two cycloalkyl or cycloheteroalkyl groups that are bound to one another. Non-limiting examples include bicyclohexane and bipiperidine.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₁₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

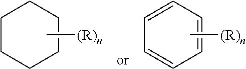

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

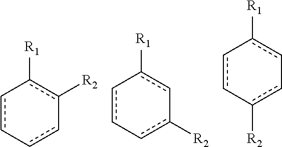

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include Ct-20 inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (H), iodine-125 (¹²I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms.

In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

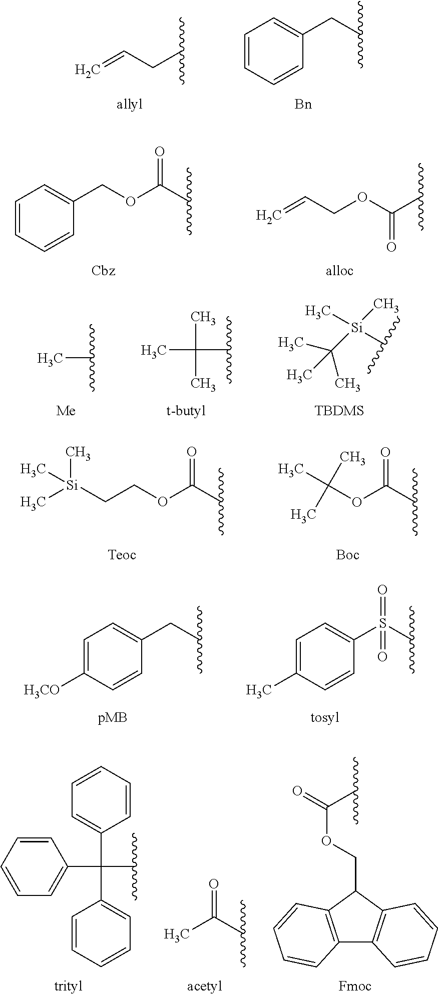

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments±20%, in some embodiments±10%, in some embodiments±5%, in some embodiments ±1%, in some embodiments±0.5%, and in some embodiments 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Pharmacokinetics and Brain Penetration of D-DOPA and Prodrugs Thereof as Novel Glutamate Carboxypeptidase (GCP-II) Inhibitors

### 1.1 Objective

In some embodiments, the presently disclosed subject matter assesses the pharmacokinetics (PK), brain penetration, and target engagement of D-DOPA and prodrugs thereof, given orally and via intranasal routes, using a validated LC/MS-MS bioanalytical method.

### 1.2 Overview

Glutamate carboxypeptidase II (GCPII) is a zinc metalloenzyme that hydrolyzes N-acetylaspartylglutamate (NAAG) to produce glutamate in the nervous system, and has emerged as a pharmacologically exploitable target. Vornov et al, 2016 Several potent and competitive GCPII inhibitors have been discovered in the past, however, owing to their polar structures, most have poor bioavailability and brain penetration Vornov et al., 2016; Dash et al., 2019: Nedelcovych et al., 2017. See also, Scheme 1.

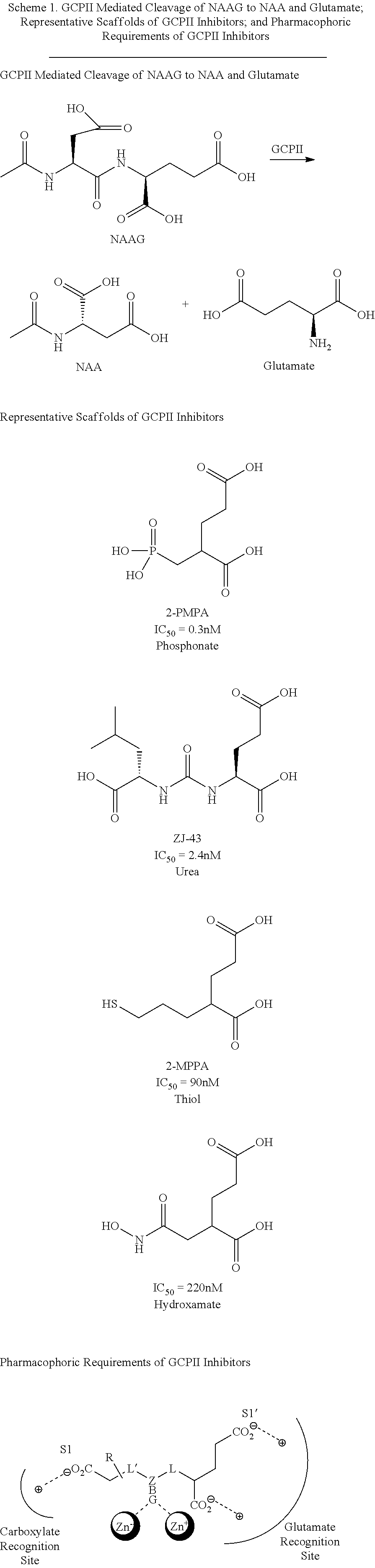

In the presently disclosed subject matter, D-3,4-dihydroxyphenylalanine (D-DOPA) was identified as a potent GCPII inhibitor (IC₅₀=200 nM), which was structurally distinct from the reported potent inhibitors, such as phosphonates (e.g., 2-(phosphonomethyl) pentanedioic acid), hydroxamates (e.g., 2-(2-(hydroxy-ainino)-2-oxoethyl)pentanedioic acid), and thiols (e.g., 2-(3-mercaptopropyl)pentanedioic acid). Limited information, however, is available on the pharmacokinetics (PK) and brain penetration of D-DOPA.

### 1.3 Methods

### 1.3.1 Analysis of D-DOPA and Prodrugs Thereof in Mouse Plasma and Brain

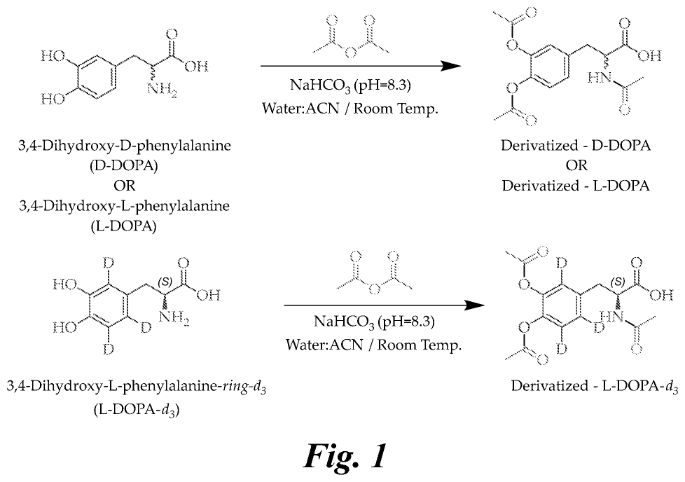

A bioanalytical method was developed to analyze D-DOPA in mouse plasma and brain using derivatization of the polar moieties with acetic anhydride (FIG. 1A)

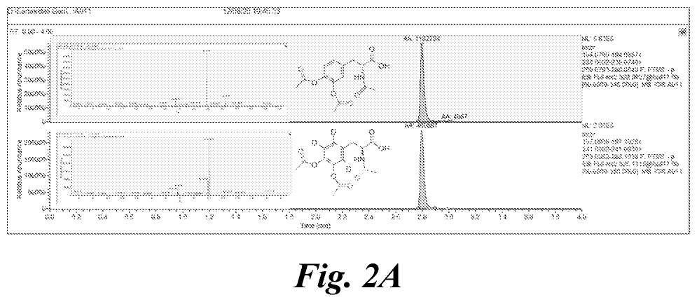

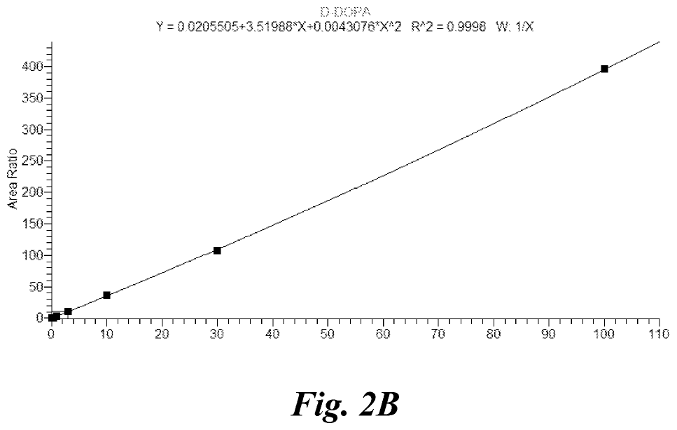

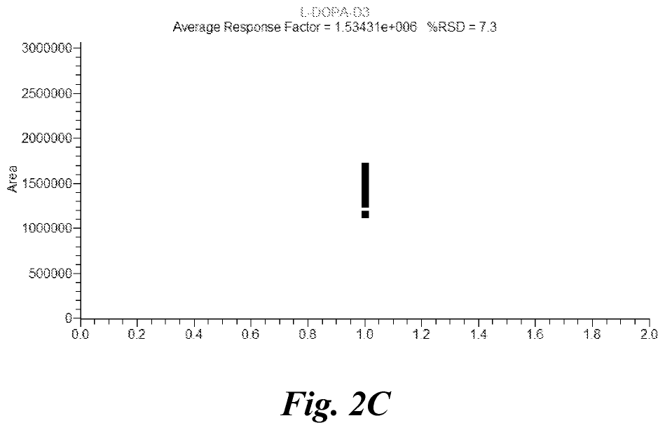

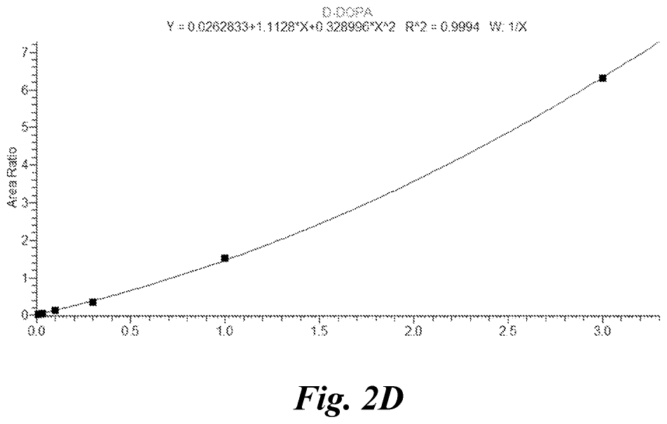

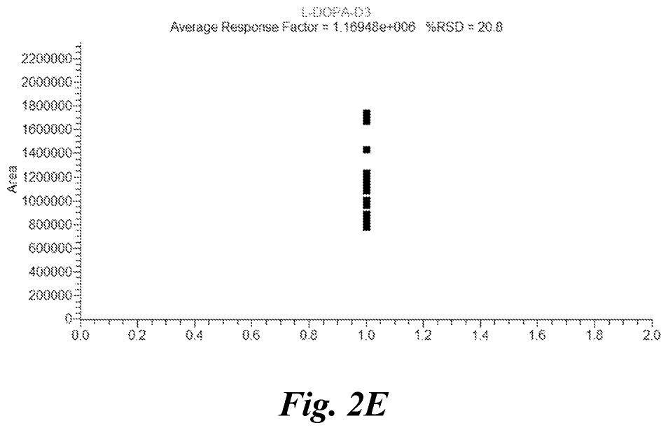

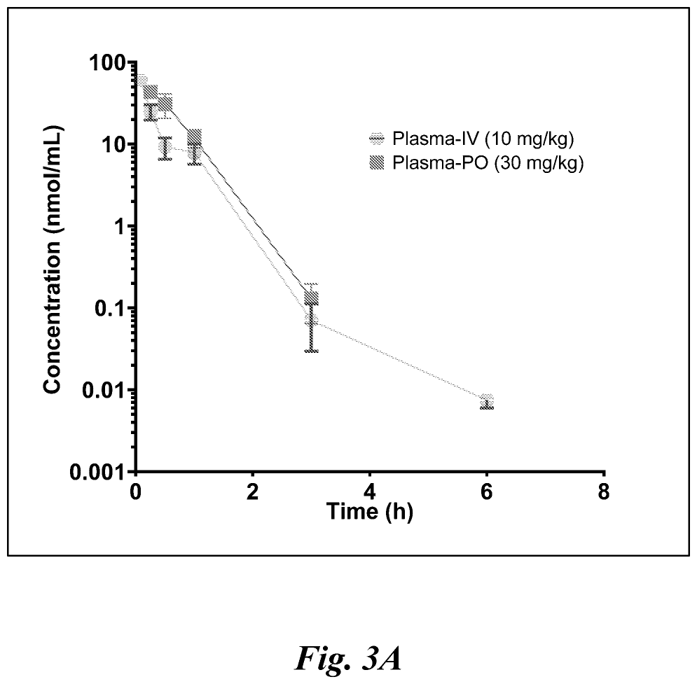

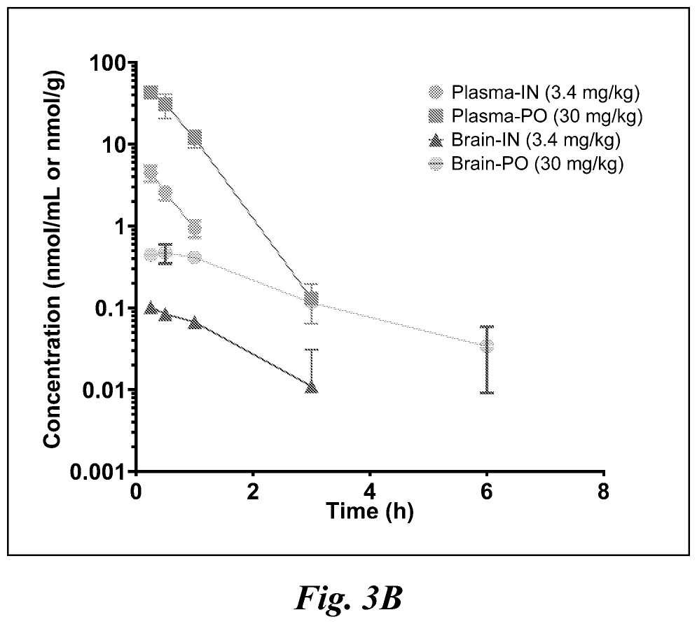

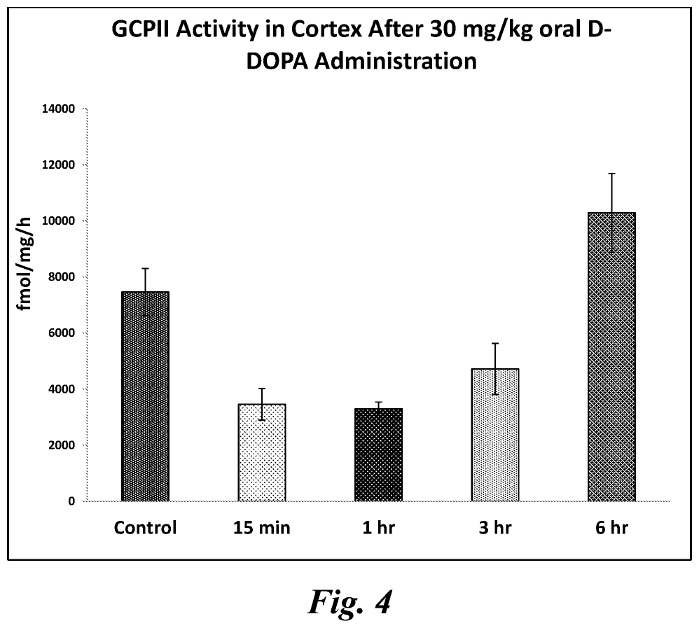

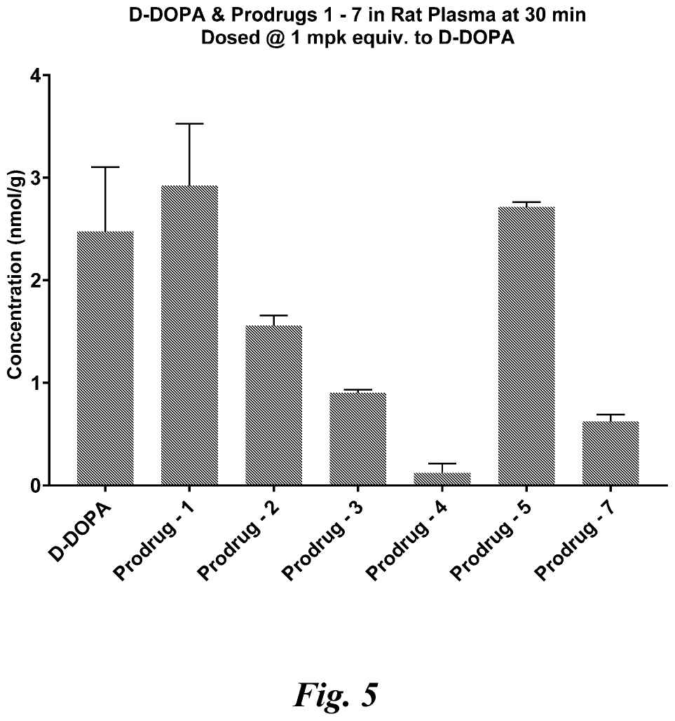

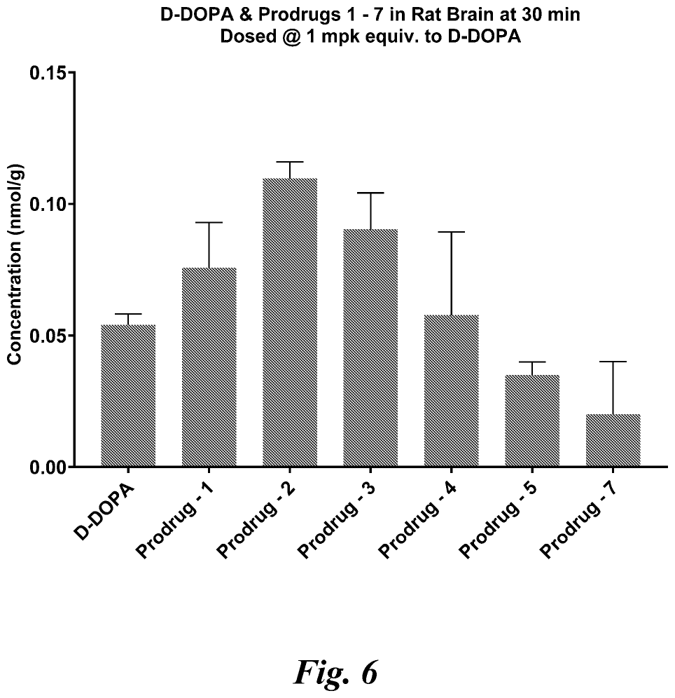

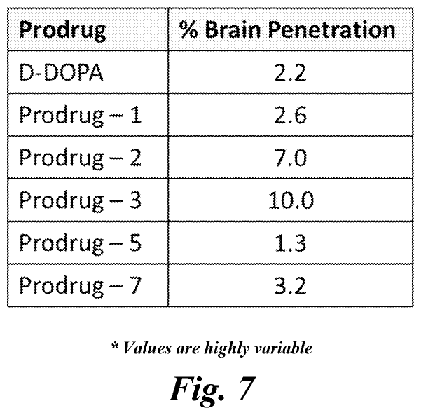

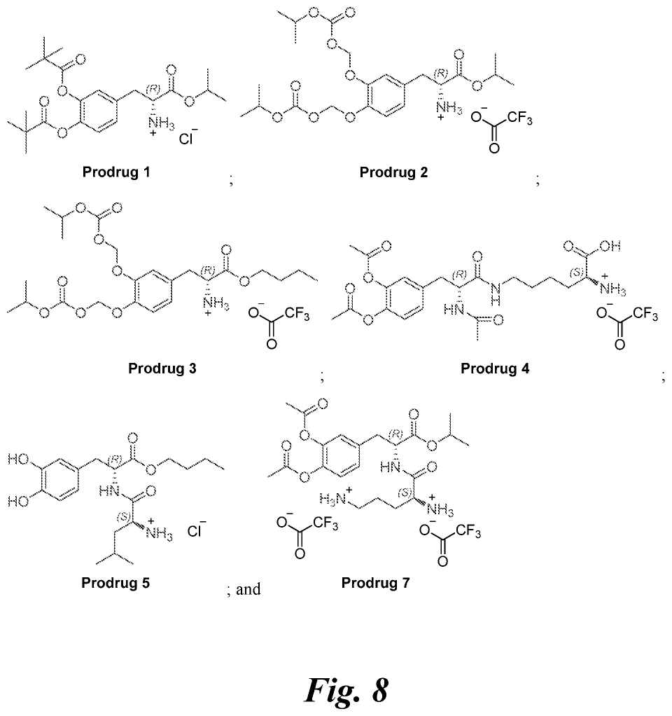

Separation of analytes was achieved using an Agilent Eclipse Plus C18 RRHD 2.1×100 mm, 1.8 μm particle size column. Samples were analyzed using UtiMate 3000 UHPLC coupled to Q Exactive Focus orbitrap mass spectrometer (FIG. 2).

The developed method was validated and employed to quamuify D-DOPA levels in plasma and brain of mice following intravenous, peroral, and intranasal dosing.

Oral availability of D-DOPA was evaluated via IV and PO dosing of D-DOPA in CD1 mice.

Brain penetration index was evaluated following oral and intranasal administration of D-DOPA in CD1 mice and measuring DOPA levels in brain and plasma.

Finally, target engagement was confirmed by measuring GCPII activity in the brain using published methods.

1.3.2 D-DOPA Rat Pharmacokinetics In_{surg}. Dosing

TABLE 2
PK Study Details

| Test compound | D-DOPA |
|---|---|
| Dose (IN) | 1 mg/kg equivalent to D-DOPA |
| Time points | 1 (30 m) |
| | n = 3 rats/timepoint |
| Matrices collected | Plasma, Brain, Olfactory Bulb |

### 1.4 Results

A quantitative LC/MS method linear over four orders of magnitude in plasma (0.01-100 nmol/mL) and three orders of magnitude in brain (0.01-3 nmol/g) with good linearity (R2≥0.9950), precision (RSDs≤25%), and accuracy (85-113%) was developed.

When dosed IV at 10 mg/kg in CD1 mice, DOPA levels reached a maximum concentration of 61.1±5.09 nmol/mL (Cₘₐₓ) in plasma within 5 min (Tₘₐₓ) with overall plasma exposure (AUC0-t) of 30.5±2.04 nmol·h/mL. When dosed PO at 30 mg/kg in CD1 mice, peak plasma DOPA levels (Cₘₐₓ) were recorded at 43.1±2.58 nmol/mL at 15 min (Tₘₐₓ) with overall plasma exposures (AUC0-t) measuring 39.92±0.731 nmol·h/mL; for this cohort, peak brain exposures (Cₘₐₓ) were recorded to be 0.472±0 124 nmol/g at 30 min (Tmax) with overall brain exposures measuring 1.09±0.103 nmol·h/g.

The oral bioavailability of D-DOPA was calculated to be 36% and brain penetration index (AUC_{brain/plasma}) was observed to be less than 5%. Intranasal administration of D-DOPA did not improve the brain penetration index in mice (AUC_{brain/plasma}=5.1%).

Target engagement studies following oral dose at 30 mg/kg revealed significant inhibition of GCPII activity in the brain at 1 h post oral administration of D-DOPA; whereas, intranasal dosing was hampered by the poor solubility of D-DOPA in vehicle resulting in insufficient exposures to the brain to affect GCP II activity.

The calculated pharmacokinetic parameters of D-DOPA in mouse plasma when dosed using different dosing routes are provided in Table 3.

TABLE 3
Pharmacokinetic parameters of D-DOPA dosed IV, PO,
and IN in mice, oral bioavailability was 36%.

| Dosing | | Pharmacokinetic Parameters | | | | |
|---|---|---|---|---|---|---|
| Route & | | Cₘₐₓ | | AUC₀₋ₜ | | |
| Dose | | (nmol/mL or | Tₘₐₓ | (nmol · h/mL or | Bioavailability | |
| Level | Matrix | nmol/g) | (h) | nmol · h/g) | (% F) | AUC_{Brain/Plasma} |
| IV | Plasma | 61.1 ± 5.09 | 0.0833 | 30.5 ± 2.04 | — | |
| 10 mg/kg | | | | | | |
| PO | Plasma | 43.1 ± 2.58 | 0.250 | 32.92 ± 0.731 | 36 | |
| 30 mg/kg | Brain | 0.472 ± 0.124 | 0.500 | 1.09 ± 0.103 | — | 3.3% |
| IN | Plasma | 4.45 ± 0.971 | 0.250 | 2.70 ± 0.508 | — | |
| 3.4 mg/kg | Brain | 0.102 ± 0.00823 | 0.250 | 0.139 ± 0.0268 | — | 5.1% |

### 1.5 Summary

These studies describe a reliable method for pharmacokinetic evaluation of D-DOPA levels in plasma and brain and support its further development as a new scaffold for synthesizing novel GCPII inhibitors. Future studies will be focused on chemical optimization of D-DOPA for enhancing its brain penetration.

### Example 2

D-DOPA is a Potent, Orally Bioavailable, Allosteric Inhibitor of Glutamate Carboxypeptidase II with Robust Target Engagement in the Brain

### 2.1 Overview

Glutamate carboxypeptidase-II (GCPII) is a zinc-dependent metalloenzyme implicated in numerous neurological disorders. The pharmacophoric requirements of active-site GCPII inhibitors makes them highly charged, manifesting poor pharmacokinetic (PK) properties. Herein, we describe the discovery and characterization of catechol-based inhibitors including L-DOPA, D-DOPA, and caffeic acid, with sub-micromolar potencies. Of these, D-DOPA emerged as the most promising compound, with good metabolic stability, and excellent PK. Orally administered D-DOPA yielded high plasma exposures (AUCₚₗₐₛₘₐ=72.7 nmol·h/mL) and an absolute oral bioavailability of 47.7%. Unfortunately, D-DOPA brain exposures were low with AUC_{brain}=2.42 nmol/g and AUC_{brain/plasma} ratio of 0.03. Given reports of isomeric inversion of D-DOPA to L-DOPA via D-amino acid oxidase (DAAO), we next evaluated D-DOPA PK in combination with the DAAO inhibitor sodium benzoate and observed a remarkable >200% enhancement in both plasma and brain exposures (AUCₚₗₐₛₘₐ=185 nmol·h/mL; AUCbᵣₐᵢₙ=5.48 nmol·h/g). Further, we demonstrated GCPII target engagement in the brain where D-DOPA+sodium benzoate combination significantly outperformed D-DOPA alone. Lastly, mode of inhibition studies revealed D-DOPA to be a noncompetitive, allosteric inhibitor of GCPII. To our knowledge, this is the first report of D-DOPA as a distinct scaffold for GCPII inhibition, laying the groundwork for future optimization to obtain clinically viable candidates.

### 2.2 Background

Glutamate carboxypeptidase II (GCPII), also known as prostate specific membrane antigen (PSMA), is a type II transmembrane metallopeptidase encoded by the folate hydrolase (FOLH1) gene in humans. O'Keefe et al., 1998. Since its discovery in 1987, Robinson et al., 1987, its expression in different tissues, such as the prostate, kidney, Chang et al., 2000, small intestine, Pinto et al., 1996; Zhao et al., 2009, and central and peripheral nervous system, Barinka et al., 2012; Guilarte et al., 2008; Yang et al., 2022, has been reported. In brain, GCPII catalyzes the hydrolysis of the neurotransmitter N-acetylaspartylglutamate (NAAG) to N-acetylaspartate (NAA) and glutamate, Ferraris et al., 2012, and multiple independent research groups have demonstrated the therapeutic benefit of inhibiting GCPII to treat neurological dysfunctions. Barinka et al., 2012; Guilarte et al., 2008; Yang et al., 2022. Similarly, perturbation in GCPII expression has been reported in cancer neovasculature, Schmidt et al., 2017, tumor angiogenesis, Conway et al., as well as in inflammatory bowel disease. Zhang et al., 2012; Ben-Schachar et al., 2013; Noble et al., 2010. GCPII inhibition is currently being explored as a therapeutic modality for these diseases. Rais et al., 2016; Evans et al., 2016; Barinka et al., 2012.

Structurally, nearly all GCPII active site inhibitors have similar pharmacophoric requirements; the scaffold consists of a zinc-binding group (ZBG), a linker, and a carboxylic acid-containing moiety designed to interact with the S1′ glutamate recognition site of the enzyme. Yang et al., 2022. The most extensively explored zinc-binding groups in the design of GCPII inhibitors include phosphonates/phosphinates, ureas, hydroxamates, and thiols. Yang et al., 2022. Given this, GCPII inhibitors designed to date are highly charged with poor PK and limited brain penetration. Vornov et al., 2016; Majer et al., 2016; Ferraris et al., 2014; Rais et al., 2017.

One strategy to overcome these limitations is to design prodrugs to mask the hydrophilic sites and thus improve bioavailability; Ferraris et al., 2014; Rais et al., 2017; Dash et al., 2019, for example, our group has reported a 5-fold improvement in rodent plasma levels of 4-carboxy-α-[3-(hydroxyamino)-3-oxopropyl]-benzenepropanoic acid, a potent hydroxamate-based GCPII inhibitor, by masking its hydrophilic hydroxamate site using para-acetoxybenzyl-esters. Rais et al., 2017. Similarly, oral administration of tetraODOL, and tris-POC prodrugs of the potent GCPII inhibitor 2-(phosphonomethyl)-pentanedioic acid (2-PMPA) yielded significantly improved plasma exposure compared to equimolar 2-PMPA in dogs and mice. Majer et al., 2016; Dash et al., 2019.

Intranasal (IN) administration of GCPII inhibitors has also been explored, specifically to improve their brain penetration index. Nedelcovych et al., 2017; Rais et al., 2015. Nedelcovych et. al. reported a consolidation of the two strategies by assessing the brain penetration of γ-(4-acetoxy benzyl) ester prodrug of 2-PMPA intranasally and showed that intranasal (IN) administration of the ester prodrug more than doubled 2-PMPA concentrations in the cerebrospinal fluid. Nedelcovych et al., 2017.

Lastly, dendrimer-based delivery systems have been employed to facilitate brain-targeted delivery of GCPII inhibitors to activated microglia. Hollinger et al., 2022; Arteaga et al., 2021. While these strategies have been somewhat promising in improving plasma and brain exposures, the search has continued for oral, brain penetrable small molecule inhibitors.

Catecholic moieties have been reported to bind to zinc metalloproteases by various groups. Veldkamp et al., 2017; Rahman et al., 2021. Therefore, in an attempt to identify novel scaffolds with good potency, oral bioavailability and good CNS penetration we evaluated known catechols as GCPII inhibitors. We screened various catechols for their ability to inhibit GCPII and identified three with submicromolar potencies.

In this example we describe evaluation of in vitro stabilities and in vivo PK of the initial compounds. Of these, D-DOPA appeared as a promising candidate with good oral PK that was enhanced when co-administered with a DAAO inhibitor, an enzyme reported to cause isomeric conversion of D-DOPA to L-DOPA. Moses et al., 1996; Wu et al., 2006; Karoum et al., 1988. We confirmed GCPII target engagement in the brain and characterized its mode of inhibition, which is distinct from current GCPII inhibitors. To our knowledge, this is the first report of a catechol-based scaffold for GCPII inhibition.

### 2.3 Materials and Methods

### 2.3.1. Reagents and Chemicals

D-DOPA, L-DOPA, caffeic acid, L-DOPA-ring-d₃, acetic anhydride, and compounds 2-3, 2-5, 2-7, 2-8, and 2-9 were purchased from Sigma-Aldrich (St. Louis, MO, USA) and used without further purification. Compounds 2-4 and 2-6 were synthesized using the previously reported methods. Kyriakou et al., 2020; Shchepin et al., 2010. LC-MS grade water, methanol, acetonitrile, and formic acid were obtained from Thermo Fisher Scientific (Waltham, MA, USA).

### 2.3.2. GCPII Activity Assay

Inhibition potencies against GCPII (IC₅₀ values) were determined using previously described methods with minor modification. Rojas et al., 2002. Briefly, reactions were carried out in the presence of NAA-[³H]-G and human recombinant GCPII enzyme in Tris-HCl and CoCl₂ at 37° C. for 20 minutes. Reactions were stopped with ice-cold sodium phosphate buffer containing 1 mM EDTA. Aliquots were then transferred to 96-well spin columns containing AG1X8 ion-exchange resin and centrifuged. NAA-[³H]-G was bound to the resin and [³H]-G eluted in the flow through. Columns were washed with formate to ensure complete elution of [³H]-G. The flow-through and the washes were collected, aliquots transferred and dried to completion in a solid scintillator-coated 96-well plate. The radioactivity corresponding to [³H]-G was determined with a scintillation counter. Subsequently, IC₅₀ curves were generated from CPM results.

### 2.3.3. Metabolic Stability

In vitro metabolic stability of L-/D-DOPA and caffeic acid was evaluated in mouse plasma and brain homogenate as we have previously described. Zimmermann et al., 2018. For the brain homogenates, washed tissues were diluted 10-fold in 0.1 M potassium phosphate buffer and homogenized using a probe sonicator. Crude homogenate and plasma were aliquoted to 1 mL and spiked with a final assay concentration of 10 μM of the respective catechol followed by incubation in an orbital shaker at 37° C. for 1 h (in triplicate). An aliquot from each incubated homogenate was quenched with three volumes of acetonitrile containing the internal standard (IS; losartan: 0.5 m) at predetermined time points (0, 30, 60 min). Samples were vortex-mixed for 30 secs and centrifuged at 10,000×g for 10 min at 4° C.

Disappearance of respective catechol was monitored on a Dionex Ultimate 3000 ultra-high-performance LC system coupled with Q Exactive Focus orbitrap mass spectrometer (Thermo Fisher Scientific Inc., Waltham MA). The separation of analytes was achieved using the Agilent Eclipse Plus column (100×2.1 mm i.d.; maintained at 35° C.) packed with a 1.8 μm C18 stationary phase. The mobile phase consisted of 0.1% formic acid in water and 0.1% formic acid in acetonitrile. Pumps were operated at a flow rate of 0.4 mL/min for 9 min using gradient elution. The mass spectrometer was controlled by Xcalibur software 4.0.27.13 (Thermo Scientific) and was operated with a heated electrospray ionization (IESI) ion source and was operated in switching ionization mode to collect both positive and negative molecular ions. Quantification of the compounds was performed in the full-scan mode (from m/z 50 to 1600) by comparing t=0 samples with t=30- and 60-min samples.

Metabolites were identified in the full-scan mode (from m/z 50-1600) by comparing t=0 samples with t=30 min samples, and structures were proposed based on the accurate mass information.

### 2.3.4. Oral Pharmacokinetic Screening in Mice

All PK studies in mice were conducted according to protocols approved by the Animal Care and Use Committee at Johns Hopkins University.

Male CD-1 mice between 25-30 g were obtained from Harlan and maintained on a 12 h light-dark cycle with ad libitum access to food and water. For in vivo PK screening, L-/D-DOPA or caffeic acid were administered perorally (PO) at a dose of 50 mg/kg (L-/D-DOPA in PBS; caffeic acid in 10% ethanol, 10% tween, 80% PBS); and a dosing volume of 10 mL/kg. All of the formulations were freshly prepared prior to the dosing. The mice were sacrificed at specified time points (30 and 60 mins) post drug administration. For the collection of plasma and brain tissue, animals were euthanized with CO₂, and blood samples were collected in heparinized microtubes by cardiac puncture. Brains were dissected and immediately flash frozen (−80° C.). Blood samples were spun at 2,000 g for 15 min, and plasma was removed and stored at −80° C. until LC-MS/MS analysis.

For time-dependent PK studies, D-DOPA was dissolved in PBS and administered either PO at a dose of 50 mg/kg or intravenously (IV; 10 mg/kg). For combination studies with sodium benzoate, a separate cohort was dosed with 400 mg/kg sodium benzoate IP 5 mins prior to D-DOPA (50 mg/kg PO). All of the formulations were freshly prepared prior to the dosing. The mice were sacrificed at specified time points (0.08, 0.25, 0.5, 1, 3, and 6 h) post drug administration. Tissue and plasma collection were carried out as described above.

### 2.3.5. Bioanalysis

Quantification of D-/L-DOPA and caffeic acid was performed using sensitive and selective methods reported in the literature with some modifications. Van Faassen et al., 2020; Wang et al., 2015. While D-/L-DOPA required derivatization with acetic anhydride, caffeic acid was analyzed using a single step protein precipitation method. For D-/L-DOPA analysis, calibration standards were prepared by spiking standard solutions of the analyte (in acetonitrile containing 0.1% formic acid) and internal standard (L-DOPA-ring-d&; 1 μM in acetonitrile containing 0.1% formic acid) in either plasma or brain extract (in acetonitrile containing 0.1% formic acid). Plasma and brain tissue samples were processed by matching the dilutions of calibration standards of the respective tissue. Plasma samples were vortex mixed and brain samples were homogenized in Geno grinder for 3 mins at 1500 cycles per minute followed by centrifugation at 10,000 g for 10 min at 4° C. to collect the supernatant. To the supernatants, sodium bicarbonate (0.2 M; pH=8.3) was added followed by derivatizing solution (acetic anhydride in equal volume acetonitrile). The solutions were vortexed for 15 secs and then allowed to sit at room temperature for 45 mins. These mixtures were vortexed again for 10 sec and centrifuged at 10,000 g for 10 mins to collect the supernatant. A 50 μL aliquot of the supernatant was transferred to 250 μL polypropylene autosampler vials sealed with Teflon caps. 2 μL of the sample was injected into the LC/MS/MS system for analysis.

For caffeic acid analysis, calibration standards were prepared by spiking standard solutions of the analyte (in methanol containing 0.5 μM losartan) into naïve plasma or brain extract (prepared in methanol containing 0.5 μM losartan). Plasma and brain tissue samples were processed by matching dilutions of the calibration standards. Plasma samples were vortex mixed and brain samples were homogenized in Geno grinder for 3 mins at 1500 cycles per minute followed by centrifugation at 10,000 g for 10 min at 4° C. to collect the supernatant. Then, 2 μL of the sample was analyzed using LC/MS/MS system as mentioned above.

Chromatographic analysis was performed using Thermo Scientific Vanquish UPLC system consisting of an analytical pump and an autosampler coupled with a TSQ Altis mass spectrometer. For all analytes, the mobile phase used for the chromatographic separation composed of 0.1% formic acid in acetonitrile and 0.1% formic acid in water delivered as a gradient while pumping a flow rate of 0.400 mL/min. Separation of the analyte was achieved using Agilent EclipsePlus C18 RRHD, 1.8 μm (2.1 mm×10 mm) column. The analyte was monitored using ThermoScientific TSQ Altis triple-quadrupole mass-spectrometric detector equipped with an electrospray interface, and operated in negative ion mode. The instrument was controlled by Thermo Scientific Xcalibur (version 4.2.47) software. The spectrometer was programmed in selected reaction monitoring (SRM) mode to monitor the transitions for L-/D-DOPA m/z 322.0→238.1, 260.0, 262.1, for L-DOPA-ring-d₃ m/z 325.0→241.1, 265.1, 283.0, for caffeic acid m/z 179.0→107.1, 135.1 and losartan m/z 421.1→127.1, 179.1.

Plasma concentrations (nmol/mL) and brain tissue concentrations (nmol/g) were determined and plots of mean plasma or brain concentrations versus time were constructed. Non-compartmental analysis modules in Phoenix WinNonlin version 7.0 (Certara USA, Inc., Princeton, NJ) were used to quantify exposures (AUC₀₋ₜ), half-life (t_{1/2}), volume of distribution (V_{d}) and clearance (Cl).

### 2.3.6. GCPII Target Engagement of D-DOPA and Mode of Inhibition Studies

GCPII activity measurements were carried out based on a modification of a previously published protocol. Sala et al., 2020. Briefly, brain samples were homogenized in ice-cold Tris buffer containing protease inhibitors. Resulting homogenates were spun down and the supernatants collected for both GCPII activity measurements and protein analysis. GCPII reaction was initiated upon the addition of homogenate, cobalt chloride and 3H-NAAG (0.04 μM, 20 mCi/μmol). Reactions were carried out in 50 μL reaction volumes for 2 h at 37° C. At the end of the incubation period, reactions were terminated with ice-cold sodium phosphate buffer and [³H]-glutamate measured as described above. Finally, total protein measurements, were carried out using BioRad's detergent compatible protein assay kit and data were presented as fmol/mg/h.

To determine mode of inhibition, experiments were carried out as described in the GCPII assay, Rojas et al., 2002, except that concentrations of NAA-[³H]-G were extended up to 4 μM and in the presence and absence of D-DOPA (0, 100, 200, 300, 400 nM). Kₘ, Vₘₐₓ calculations were determined using Michelis-Menten kinetic analysis using GraphPad Prism (version 9.3.0).

### 2.4. Results and Discussion

### 2.4.1 Identification of Catechol-Based GCPII Inhibitors

The inhibitory potencies of the catechols were determined using a modified radioactivity-based assay involving human recombinant GCPII enzyme as previously reported. Rojas et al., 2002. Based on literature reports of catecholic moieties binding to zinc metalloproteases, Veldkamp et al., 2017; Rahman et al., 2021, we tested the potency of L-DOPA (1) against GCPII activity and discovered that it is a submicromolar inhibitor of GCPII. Although its potency is weaker than those of known GCPII inhibitors containing multiple carboxylate groups, L-DOPA represents one of the most potent monocarboxylate-based GCPII inhibitors. This prompted us to evaluate analogs of L-DOPA (Table 2-1) to determine the essential structural features responsible for its GCPII inhibitory activity. Both removal of the meta-hydroxy group and methylation of the two hydroxy groups resulted in substantial loss of potency as seen in compounds 2-2 and 2-3. Replacement of the catechol moiety with a 2-pyridone (compound 2-4) also led to complete loss of potency. These findings suggest that the catechol moiety of L-DOPA is essential for the GCPII inhibitory activity. Subsequently, we tested L-DOPA analogs retaining the catechol moiety (compounds 2-5, 2-6, 2-7, 2-8, 2-9, and 2-10). While alpha-methylation (compound 2-5), N-acetylation (compound 2-6), decarboxylation (compound 2-7) resulted in loss of potency, the removal of the alpha-amino group (compound 2-8) led to only 6.7-fold decrease in inhibitory potency. Interestingly, conversion of the alpha-beta single bond to a double bond (compound 2-9, caffeic acid) generated another potent GCPII inhibitor among the L-DOPA analogs with an IC₅₀ value of 300 nM. Finally, D-DOPA, an enantiomer of L-DOPA, was found to be the most potent catechol with an IC₅₀ value of 200 nM.

Taken together, these structure activity relationship (SAR) studies highlight the critical role played by the catechol and carboxylate groups of L-DOPA in GPCII inhibition, as well as the insignificant contribution of the alpha amino group. We chose to further assess the three submicromolar inhibitors (L-DOPA, caffeic acid, and D-DOPA) for their potential to serve as in vivo pharmacological probes for GCPII inhibition.

TABLE 2-1
The comparative IC₅₀ of catechols determined using human recombinant
GCPII. D-DOPA, L-DOPA, and caffeic acid were advanced for in vitro/
in vivo screening based on their superior potency over other catechols
tested.

| Compound | Structure | IC₅₀ (μM) |
|---|---|---|
| 2-1 (L-DOPA) | 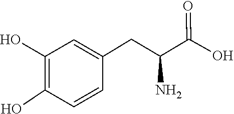 [SMILES: N[C@@H](Cc1ccc(O)c(O)c1)C(=O)O] | 0.6 |
| 2-2 (Tyr) | 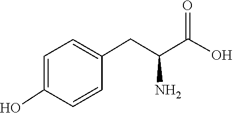 [SMILES: N[C@@H](Cc1ccc(O)cc1)C(=O)O] | 100 |
| 2-3 | 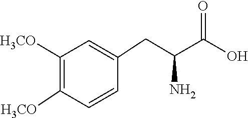 [SMILES: COc1ccc(C[C@H](N)C(=O)O)cc1OC] | >100 |
| 2-4 | 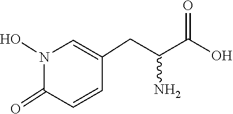 [SMILES: NC(Cc1ccc(=O)n(O)c1)C(=O)O] | >100 |
| 2-5 | 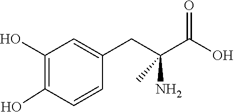 | >100 |
| 2-6 | 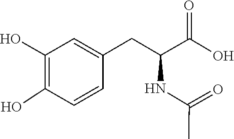 [SMILES: CC(=O)N[C@@H](Cc1ccc(O)c(O)c1)C(=O)O] | 20 |
| 2-7 (Dopamine) | 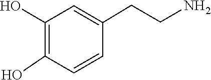 [SMILES: NCCc1ccc(O)c(O)c1] | >100 |
| 2-8 | 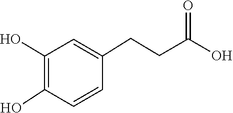 [SMILES: O=C(O)CCc1ccc(O)c(O)c1] | 4 |
| 2-9 (Caffeic Acid) | 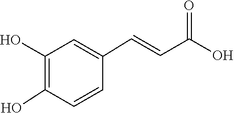 [SMILES: O=C(O)/C=C/c1ccc(O)c(O)c1] | 0.3 |
| 2-10 (D-DOPA) | 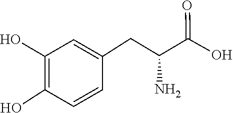 [SMILES: N[C@H](Cc1ccc(O)c(O)c1)C(=O)O] | 0.2 |

### 2.4.2 Bioanalytical Methods for D-L-DOPA and Caffeic Acid

To conduct in vivo assessment of selected catechols we searched literature reports for facile and sensitive LC-MS methods. Caffeic acid resolved well on reverse phase column and was detectable on MS with low nM sensitivity; however, reported methods for DOPA analyses either required complex sample preparation, such as solid phase extraction, Li et al., 2000, or used harsh acidic conditions (e.g., 0.4 M perchloric acid) not suitable for UPLC-MS. Li et al., 2000; Igarashi et al., 2003. Although many methods have been reported for DOPA analysis in plasma, few have evaluated brain levels. We thus employed a derivatization method previously reported by van Faassen et al., with some modifications as needed. Van Faassen et al., 2020. We used acetic anhydride in the presence of sodium bicarbonate (pH=8.3; 0.2 M) to acetylate the polar catecholic and amino groups thus improving the lipophilicity of DOPA for better retention on reverse phase column. For DOPA analyses, we used L-DOPA-ring-d₃ as internal standard. DOPA and L-DOPA-ring-d₃ were derivatized at three sites; including, both phenolic sites as well as the primary amine of the amino acid. Li et al., 2000.

The lower limit of quantification (LLOQ) for DOPA in plasma and brain was observed to be 0.03 nmol/mL and 0.10 nmol/g respectively; similarly, the LLOQ for caffeic acid in plasma and brain was observed to be 0.01 nmol/mL and 0.01 nmol/g respectively. These were similar/better than literature reports of LLOQ of L-DOPA in rat plasma, that varies from 0.02 to 0.15 nmol/mL, Cho et al., 2012; Lv et al., 2010, whereas for caffeic acid it is reported to be 0.03 nmol/mL. Wang et al., 2015.

Correlation coefficient of >0.99 was obtained in all analytical runs and internal standard variation of <15% was observed for all analyses. The mean-predicted concentration accuracy for calibration standards ranged from 85 to 111% for caffeic acid, 86 to 116% for D-DOPA and 97 to 111% for L-DOPA. For quality control samples, the mean-predicted accuracy ranged from 86 to 98% for caffeic acid, 94 to 115% for D-DOPA, and 86 to 113% for L-DOPA.

### 2.4.3 Mouse Two-Point Pharmacokinetic Screening Studies of D-L-DOPA and Caffeic Acid

Clearance of L-DOPA is expected to occur primarily via metabolism to dopamine during first pass metabolism. De Vries et al., 1992; Vieira-Coelho and Soares-da-Silva, 1993. To circumvent this, decarboxylase inhibitors such as carbidopa are routinely employed in combination with L-DOPA, Muller et al., 2020, and abundant evidence exists supporting higher peripheral DOPA exposure when L-DOPA/carbidopa are co-administered. Rose et al., 1991. While this is an efficient solution to improve peripheral exposure of L-DOPA, co-administration of carbidopa may not impact its brain metabolism, and thus D-DOPA was preferable scaffold for GCPII inhibition in the brain, due to its greater metabolic stability and PK profile in comparison to L-DOPA. Finally, the low plasma levels of caffeic acid (6-7 nmol/mL) are consistent with its widely reported poor oral bioavailability and low intestinal absorption, Wang et al., 2014. Further the hydrophilicity of these compounds limits their brain penetration as reflected in this in vivo screening study. Overall, based on the initial screening results showing 3-fold higher potency versus L-DOPA, superior in vitro stability, and higher plasma and brain exposure, we advanced D-DOPA to further assess its time-dependent PK properties.

### 2.4.4 Mouse Pharmacokinetic and Target Engagement Studies of D-DOPA+/−Sodium Benzoate

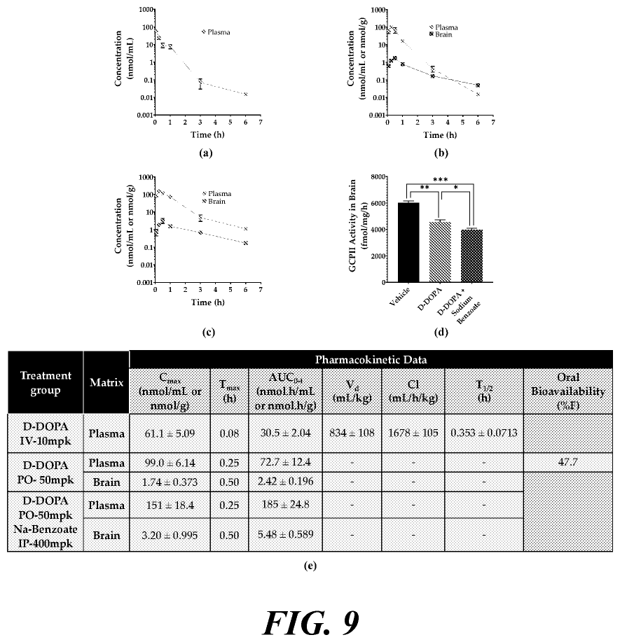

Time-dependent PK profiles of D-DOPA in mouse plasma and brain are presented in FIG. 9a, FIG. 9b, and FIG. 9c; and detailed PK parameters are presented in FIG. 9e. After a single IV dose of 10 mg/kg, D-DOPA achieved a maximum concentration (Cₘₐₓ) of 61.1 nmol/mL in plasma at 5 min (Tₘₐₓ) (FIG. 9a). The half-life (t_{1/2}), volume of distribution (V_{d}), and clearance (Cl) of D-DOPA in plasma were calculated to be 0.35 h, 0.834 L/kg, and 28 mL/min/kg respectively. The overall exposures in plasma (AUC_{IV}) were calculated to be 30.5 nmol·h/mL (FIG. 9e). After a single PO dose (50 mg/kg), D-DOPA achieved a Cₘₐₓ of 99.0 nmol/mL in plasma at 15 min and 1.74 nmol/mL in brain at 30 min (FIG. 9b). AUC_{PO-plasma} and AUC_{PO-Brain} were calculated to be 72.7 nmol·h/mL and 2.42 nmol·h/g, respectively. D-DOPA also exhibited excellent oral bioavailability at 47.7% (AUC_{PO/IV}). The brain penetration index was somewhat low with AUC_{Brain/Plasma}=0.033; although the concentrations up to 1 h time point were >IC₅₀. Several groups have reported a caveat that D-DOPA is unidirectionally converted to its L-isomer in vivo. Moses et al., 1996; Wu et al., 2006. The chiral inversion of D-DOPA to L-DOPA is facilitated by oxidative deamination of D-DOPA by DAAO; the resulting alpha-keto acid is then transaminated to L-DOPA by dopa transaminase. Thus, we strategized to evaluate the PK of D-DOPA when co-administered with a DAAO inhibitor as a mechanism to enhance its plasma and brain exposures.

Mice were dosed IP with 400 mg/kg sodium benzoate, followed by D-DOPA (50 mg/kg PO). D-DOPA achieved 1.53-fold higher Cₘₐₓ (151 nmol/mL) in plasma at 15 min and a 1.84-fold higher Cₘₐₓ in brain (3.20 nmol/g at 30 min) compared to monotherapy (FIG. 9b and FIG. 9c). Furthermore, plasma exposures (AUC_{Plasma}) remarkably improved by 2.54-fold (185 nmol·h/mL; *p<0.05) and AUC_{Brain} by 2.26-fold (5.48 nmol·h/g; *p<0.05). Similar modulation in the PK of D-serine, a substrate of DAAO, has been reported by our group and others where combination with a DAAO inhibitor led to decreased clearance and increased D-serine exposures. Rais et al., 2012; Ferraris et al., 2008. This improved exposure further enhanced the oral bioavailability (47.7%) of D-DOPA to >100% and was significantly better than contemporary active site GCPII scaffolds that show <5% bioavailability, van der Post et al., 2005; Vornov et al., 2013, with the exception of thiol-based GCPII inhibitor that exhibit approximately 30-40% oral bioavailability in preclinical species. Rais et al., 2012. The brain levels of D-DOPA, although low, stayed >IC₅₀ value of 0.2 μM for over 3 h while maintaining the brain penetration index at 0.03 (FIG. 9c). Lastly, we confirmed target engagement in brain samples from D-DOPA mono-therapy (Cₘₐₓ=1.74 nmol/g) and combination therapy (Cₘₐₓ=3.2 nmol/g), by evaluating effects on GCPII activity. Sala et al., 2020. Both treatment groups showed a significant inhibition of GCPII activity; moreover, D-DOPA co-administered with sodium benzoate significantly outperformed D-DOPA mono-therapy (***p<0.001—untreated vs. combination therapy, **p<0.01—untreated vs. monotherapy; *p<0.05—monotherapy vs. combination therapy) corroborating the PK results (FIG. 9d).

### 2.4.5 Characterization of D-DOPA's Mode of GCPII Inhibition

Most GCPII inhibitors designed to date contain pharmacophoric features for active site binding; viz., phosphonic acid-based, Jackson et al., 2001; Jackson et al., 1996, thiol-based, Majer et al., 2003, urea-based, Kozikowski et al., 2004, and hydroxamic acid-based. Stoermer et al., 2003. As discussed earlier, our initial premise for exploring the inhibition potencies of catechols against GCPII activity was their propensity for zinc binding in other similar metalloproteases. Rahman et al., 2021. Considering the structural differences between contemporary inhibitors of GCPII versus catechols, we thought it would be prudent to verify the mode of inhibition.

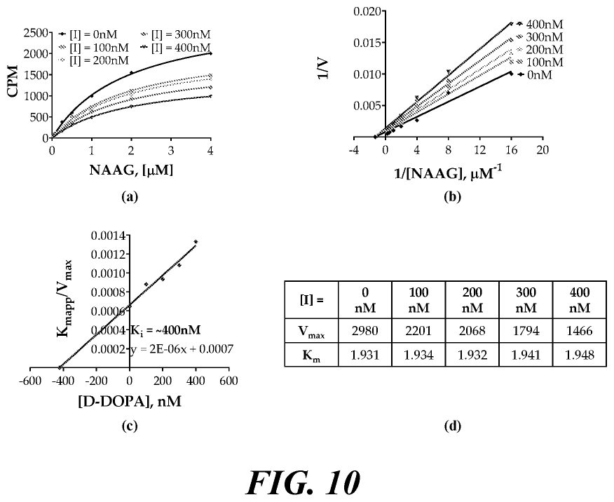

To evaluate this, NAAG saturation experiments were performed in presence of different concentrations of D-DOPA (FIG. 10a). When the rate of reaction was plotted against NAAG concentrations at increasing inhibitor concentrations, there was a decrease in maximal rate (Vₘₐₓ) while the Michaelis constant (Kₘ) was unchanged (FIG. 10a). Vₘₐₓ and Kₘ for each dataset at a given inhibitor concentration were obtained from non-linear regression fits to Michaelis-Menten kinetics (FIG. 10d). A double reciprocal plot (Lineweaver-Burk plot) of the data yielded lines with varying slopes that intersected in the second quadrant (FIG. 10b), indicative of non-competitive inhibition. A secondary plot of Kₘ apparent/Vₘₐₓ versus [D-DOPA] gave a Kᵢ value of approximately 400 nM (FIG. 10c). While the covalent binding of catechols, including L-DOPA and its analogs, to proteins via sulphahydryl interactions have been reported, this is the first description of a sub-micromolar, non-competitive inhibitor of GCPII. Thus, the mode of inhibition studies disproved our initial hypothesis and revealed D-DOPA to be an allosteric inhibitor of GCPII. Further, Parellada et. al. tested various catechols and reported that the phenolic hydroxyl groups do not coordinate with the catalytic zinc of active site in zinc metallopeptidases. Parellada et al., 1998. This report further supports the allosteric, non-competitive mode of inhibition demonstrated by our studies.

### 2.6 Summary

Small molecule GCPII inhibitors containing glutamate-mimetics have been used as a treatment for neurological disorders in preclinical models. Rais et al., 2016; Majer et al., 2003; Kozikowski et al., 2004; Rahn et al., 2012; Slusher et al., 1999; Yamamoto et al., 2001a; Yamamoto et al., 2001b; Chen et al., 2002; Carpenter et al., 2003; Ghadge et al., 2003; Ghose et al., 2009; Vornov et al., 2020. Their therapeutic potential in the clinic, however, has been hampered, in part, due to poor oral bioavailability and negligible brain penetration. We assessed known catechols and identified three with submicromolar potencies for GCPII inhibition. This is the first report of catechols as GCPII inhibitors. D-DOPA emerged as the most promising catechol-based inhibitor, demonstrating a non-competitive mode of inhibition and an excellent PK profile, which was enhanced by co-administration with the DAAO inhibitor sodium benzoate, resulting in robust target engagement in the brain. To our knowledge this is the first systematic assessment of a submicromolar, catechol-based, allosteric inhibitor of GCPII.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
O'Keefe, D. S.; Su, S. L.; Bacich, D. J.; Horiguchi, Y.; Luo, Y.; Powell, C. T.; Zandvliet, D.; Russell, P. J.; Molloy, P. L.; Nowak, N. J.; et al. Mapping, genomic organization and promoter analysis of the human prostate-specific membrane antigen gene. Biochim Biophys Acta 1998, 1443, 113-127.
Robinson, M. B.; Blakely, R. D.; Couto, R.; Coyle, J. T. Hydrolysis of the brain dipeptide N-acetyl-L-aspartyl-L-glutamate. Identification and characterization of a novel N-acetylated alpha-linked acidic dipeptidase activity from rat brain. J Biol Chem 1987, 262, 14498-14506.
Chang, S. S.; Gaudin, P. B.; Reuter, V. E.; Heston, W. D. Prostate-specific membrane antigen: present and future applications. Urology 2000, 55, 622-629.
Pinto, J. T.; Suffoletto, B. P.; Berzin, T. M.; Qiao, C. H.; Lin, S.; Tong, W. P.; May, F.; Mukherjee, B.; Heston, W. D. Prostate-specific membrane antigen: a novel folate hydrolase in human prostatic carcinoma cells. Clin Cancer Res 1996, 2, 1445-1451.
Zhao, R.; Matherly, L. H.; Goldman, I. D. Membrane transporters and folate homeostasis: intestinal absorption and transport into systemic compartments and tissues. Expert Rev Mol Med 2009, 11, e4.
Barinka, C.; Rojas, C.; Slusher, B.; Pomper, M. Glutamate carboxypeptidase II in diagnosis and treatment of neurologic disorders and prostate cancer. Curr Med Chem 2012, 19, 856-870.
Guilarte, T. R.; Hammoud, D. A.; McGlothan, J. L.; Caffo, B. S.; Foss, C. A.; Kozikowski, A. P.; Pomper, M. G. Dysregulation of glutamate carboxypeptidase II in psychiatric disease. Schizophr Res 2008, 99, 324-332.
Yang, S.; Datta, D.; Elizabeth, W.; Duque, A.; Morozov, Y. M.; Arellano, J.; Slusher, B. S.; Wang, M.; Arnsten, A. F. T. Inhibition of glutamate-carboxypeptidase-II in dorsolateral prefrontal cortex: potential therapeutic target for neuroinflammatory cognitive disorders. Mol Psychiatry 2022.
Ferraris, D. V.; Shukla, K.; Tsukamoto, T. Structure-activity relationships of glutamate carboxypeptidase II (GCPII) inhibitors. Curr Med Chem 2012, 19, 1282-1294.
Schmidt, L. H.; Heitkotter, B.; Schulze, A. B.; Schliemann, C.; Steinestel, K.; Trautmann, M.; Marra, A.; Hillejan, L.; Mohr, M.; Evers, G.; et al. Prostate specific membrane antigen (PSMA) expression in non-small cell lung cancer. PLoS One 2017, 12, e0186280.
Conway, R. E.; Petrovic, N.; Li, Z.; Heston, W.; Wu, D.; Shapiro, L. H. Prostate-specific membrane antigen regulates angiogenesis by modulating integrin signal transduction. Mol Cell Biol 2006, 26, 5310-5324.
Zhang, T.; Song, B.; Zhu, W.; Xu, X.; Gong, Q. Q.; Morando, C.; Dassopoulos, T.; Newberry, R. D.; Hunt, S. R.; Li, E. An ileal Crohn's disease gene signature based on whole human genome expression profiles of disease unaffected ileal mucosal biopsies. PLoS One 2012, 7, e37139.
Ben-Shachar, S.; Yanai, H.; Baram, L.; Elad, H.; Meirovithz, E.; Ofer, A.; Brazowski, E.; Tulchinsky, H.; Pasmanik-Chor, M.; Dotan, I. Gene expression profiles of ileal inflammatory bowel disease correlate with disease phenotype and advance understanding of its immunopathogenesis. Inflamm Bowel Dis 2013, 19, 2509-2521.
Noble, C. L.; Abbas, A. R.; Lees, C. W.; Cornelius, J.; Toy, K.; Modrusan, Z.; Clark, H. F.; Arnott, I. D.; Penman, I. D.; Satsangi, J.; et al. Characterization of intestinal gene expression profiles in Crohn's disease by genome-wide microarray analysis. Inflamm Bowel Dis 2010, 16, 1717-1728.
Rais, R.; Jiang, W.; Zhai, H.; Wozniak, K. M.; Stathis, M.; Hollinger, K. R.; Thomas, A. G.; Rojas, C.; Vornov, J. J.; Marohn, M.; et al. FOLH1/GCPII is elevated in IBD patients, and its inhibition ameliorates murine IBD abnormalities. JCI Insight 2016, 1.
Evans, J. C.; Malhotra, M.; Cryan, J. F.; O'Driscoll, C. M. The therapeutic and diagnostic potential of the prostate specific membrane antigen/glutamate carboxypeptidase II (PSMA/GCPII) in cancer and neurological disease. Br J Pharmacol 2016, 173, 3041-3079.
Barinka, C.; Rojas, C.; Slusher, B.; Pomper, M. Glutamate Carboxypeptidase II in Diagnosis and Treatment of Neurologic Disorders and Prostate Cancer. Current Medicinal Chemistry 2012, 19, 856-870.
Vornov, J. J.; Hollinger, K. R.; Jackson, P. F.; Wozniak, K. M.; Farah, M. H.; Majer, P.; Rais, R.; Slusher, B. S. Chapter Nine—Still NAAG'ing After All These Years: The Continuing Pursuit of GCPII Inhibitors. In Advances in Pharmacology, Schwarcz, R., Ed.; Academic Press: 2016; Volume 76, pp. 215-255.
Majer, P.; Jancarik, A.; Krecmerova, M.; Tichy, T.; Tenora, L.; Wozniak, K.; Wu, Y.; Pommier, E.; Ferraris, D.; Rais, R.; et al. Discovery of Orally Available Prodrugs of the Glutamate Carboxypeptidase II (GCPII) Inhibitor 2-Phosphonomethylpentanedioic Acid (2-PMPA). J Med Chem 2016, 59, 2810-2819.
Ferraris, D. V.; Majer, P.; Ni, C.; Slusher, C. E.; Rais, R.; Wu, Y.; Wozniak, K. M.; Alt, J.; Rojas, C.; Slusher, B. S.; et al. delta-Thiolactones as prodrugs of thiol-based glutamate carboxypeptidase II (GCPII) inhibitors. J Med Chem 2014, 57, 243-247.
Rais, R.; Vavra, J.; Tichy, T.; Dash, R. P.; Gadiano, A. J.; Tenora, L.; Monincova, L.; Barinka, C.; Alt, J.; Zimmermann, S. C.; et al. Discovery of a para-Acetoxy-benzyl Ester Prodrug of a Hydroxamate-Based Glutamate Carboxypeptidase II Inhibitor as Oral Therapy for Neuropathic Pain. J Med Chem 2017, 60, 7799-7809.
Dash, R. P.; Tichy, T.; Veeravalli, V.; Lam, J.; Alt, J.; Wu, Y.; Tenora, L.; Majer, P.; Slusher, B. S.; Rais, R. Enhanced Oral Bioavailability of 2-(Phosphonomethyl)-pentanedioic Acid (2-PMPA) from its (5-Methyl-2-oxo-1,3-dioxol-4-yl)methyl (ODOL)-Based Prodrugs. Mol Pharm 2019, 16, 4292-4301.
Nedelcovych, M.; Dash, R. P.; Tenora, L.; Zimmermann, S. C.; Gadiano, A. J.; Garrett, C.; Alt, J.; Hollinger, K. R.; Pommier, E.; Jancarik, A.; et al. Enhanced Brain Delivery of 2-(Phosphonomethyl)pentanedioic Acid Following Intranasal Administration of Its gamma-Substituted Ester Prodrugs. Mol Pharm 2017, 14, 3248-3257.
Rais, R.; Wozniak, K.; Wu, Y.; Niwa, M.; Stathis, M.; Alt, J.; Giroux, M.; Sawa, A.; Rojas, C.; Slusher, B. S. Selective CNS Uptake of the GCP-II Inhibitor 2-PMPA following Intranasal Administration. PLoS One 2015, 10, e0131861.
Hollinger, K. R.; Sharma, A.; Tallon, C.; Lovell, L.; Thomas, A. G.; Zhu, X.; Wiseman, R.; Wu, Y.; Kambhampati, S. P.; Liaw, K.; et al. Dendrimer-2PMPA selectively blocks upregulated microglial GCPII activity and improves cognition in a mouse model of multiple sclerosis. Nanotheranostics 2022, 6, 126-142.
Arteaga Cabeza, O.; Zhang, Z.; Smith Khoury, E.; Sheldon, R. A.; Sharma, A.; Zhang, F.; Slusher, B. S.; Kannan, R. M.; Kannan, S.; Ferriero, D. M. Neuroprotective effects of a dendrimer-based glutamate carboxypeptidase inhibitor on superoxide dismutase transgenic mice after neonatal hypoxic-ischemic brain injury. Neurobiol Dis 2021, 148, 105201.
Veldkamp, K. L.; Tubergen, P. J.; Swartz, M. A.; DeVries, J. T.; Tatko, C. D. Zinc binding with L-dopa peptides. Inorg Chim Acta 2017, 461, 120-126.
Rahman, F.; Nguyen, T. M.; Adekoya, O. A.; Campestre, C.; Tortorella, P.; Sylte, I.; Winberg, J. O. Inhibition of bacterial and human zinc-metalloproteases by bisphosphonate- and catechol-containing compounds. J Enzyme Inhib Med Chem 2021, 36, 819-830.
Moses, J.; Siddiqui, A.; Silverman, P. B. Sodium benzoate differentially blocks circling induced by D- and L-dopa in the hemi-parkinsonian rat. Neurosci Lett 1996, 218,
Wu, M.; Zhou, X. J.; Konno, R.; Wang, Y. X. D-dopa is unidirectionally converted to L-dopa by D-amino acid oxidase, followed by dopa transaminase. Clin Exp Pharmacol Physiol 2006, 33, 1042-1046.
Karoum, F.; Freed, W. J.; Chuang, L. W.; Cannon-Spoor, E.; Wyatt, R. J.; Costa, E. D-dopa and L-dopa similarly elevate brain dopamine and produce turning behavior in rats. Brain Res 1988, 440, 190-194.
Kyriakou, S.; Mitsiogianni, M.; Mantso, T.; Cheung, W.; Todryk, S.; Veuger, S.; Pappa, A.; Tetard, D.; Panayiotidis, M. I. Anticancer activity of a novel methylated analogue of L-mimosine against an in vitro model of human malignant melanoma. Invest New Drugs 2020, 38, 621-633.
Shchepin, R.; Moller, M. N.; Kim, H. Y.; Hatch, D. M.; Bartesaghi, S.; Kalyanaraman, B.; Radi, R.; Porter, N. A. Tyrosine-lipid peroxide adducts from radical termination: para coupling and intramolecular Diels-Alder cyclization. J Am Chem Soc 2010, 132, 17490-17500.
Rojas, C.; Frazier, S. T.; Flanary, J.; Slusher, B. S. Kinetics and inhibition of glutamate carboxypeptidase II using a microplate assay. Anal Biochem 2002, 310, 50-54.
Zimmermann, S. C.; Tichy, T.; Vavra, J.; Dash, R. P.; Slusher, C. E.; Gadiano, A. J.; Wu, Y.; Jancarik, A.; Tenora, L.; Monincova, L.; et al. N-Substituted Prodrugs of Mebendazole Provide Improved Aqueous Solubility and Oral Bioavailability in Mice and Dogs. J Med Chem 2018, 61, 3918-3929.
van Faassen, M.; Bischoff, R.; Eijkelenkamp, K.; de Jong, W. H. A.; van der Ley, C. P.; Kema, I. P. In Matrix Derivatization Combined with LC-MS/MS Results in Ultrasensitive Quantification of Plasma Free Metanephrines and Catecholamines. Anal Chem 2020, 92, 9072-9078.
Wang, X.; Li, W.; Ma, X.; Chu, Y.; Li, S.; Guo, J.; Jia, Y.; Zhou, S.; Zhu, Y.; Liu, C. Simultaneous determination of caffeic acid and its major pharmacologically active metabolites in rat plasma by LC-MS/MS and its application in pharmacokinetic study. Biomed Chromatogr 2015, 29, 552-559.
Sala, M.; Hollinger, K. R.; Thomas, A. G.; Dash, R. P.; Tallon, C.; Veeravalli, V.; Lovell, L.; Kogler, M.; Hrebabecky, H.; Prochazkova, E.; et al. Novel Human Neutral Sphingomyelinase 2 Inhibitors as Potential Therapeutics for Alzheimer's Disease. J Med Chem 2020, 63, 6028-6056.
Maruyama, W.; Naoi, M.; Narabayashi, H. The metabolism of L-DOPA and L-threo-3,4-dihydroxyphenylserine and their effects on monoamines in the human brain: Analysis of the intraventricular fluid from parkinsonian patients. J Neurol Sci 1996, 139, 141-148.
Meiser, J.; Weindl, D.; Hiller, K. Complexity of dopamine metabolism. Cell Commun Signal 2013, 11, 34.
Peaston, R. T.; Weinkove, C. Measurement of catecholamines and their metabolites. Ann Clin Biochem 2004, 41, 17-38.
Li, W. L.; Rossi, D. T.; Fountain, S. T. Development and validation of a semi-automated method for L-dopa and dopamine in rat plasma using electrospray LC/MS/MS. J Pharmaceut Biomed 2000, 24, 325-333.
Igarashi, K.; Hotta, K.; Kasuya, F.; Abe, K.; Sakoda, S. Determination of cabergoline and L-dopa in human plasma using liquid chromatography-tandem mass spectrometry. J Chromatogr B Analyt Technol Biomed Life Sci 2003, 792, 55-61.
Cho, Y. A.; Park, S.; Seo, O. N.; Jeong, S. W.; Lee, W.-K.; Kim, C. Y.; Kim, S. T.; Cho, M. J.; Shin, S. C. Development and validation of an LC-ESI-MS/MS method for simultaneous determination of levodopa, dopamine, L-α-methyldopa and 3-O-methyldopa in rat plasma. Journal of Pharmaceutical Investigation 2012, 42, 361-368.
Lv, L.; Jiang, W. Z.; Zhou, S. Y.; Huang, X. Z.; Shi, X. X.; Lv, C., Wu, L. L.; Xu, C. Y. LC-MS-MS Simultaneous Determination of 1-Dopa and Its Prodrug 1-Dopa n-Pentyl Ester Hydrochloride in Rat Plasma. Chromatographia 2010, 72, 239-243. de Vries, M. H.; Hamelijnck, M. A.; Hofman, G. A.; Koster, A. S.; Noordhoek, J.
Decarboxylation of L-dopa in the rat isolated vascularly perfused small intestine: contribution to systemic elimination and dose-dependent first pass effect. J Pharm Pharmacol 1992, 44, 311-314
Vieira-Coelho, M. A.; Soares-da-Silva, P. Dopamine formation, from its immediate precursor 3,4-dihydroxyphenylalanine, along the rat digestive tract. Fundam Clin Pharmacol 1993, 7, 235-243.
Müller, T. Pharmacokinetics and pharmacodynamics of levodopa/carbidopa cotherapies for Parkinson's disease. Expert Opinion on Drug Metabolism & Toxicology 2020, 16, 403-414.
Rose, S.; Jenner, P.; Marsden, C. D. Peripheral pharmacokinetic handling and metabolism of L-dopa in the rat: the effect of route of administration and carbidopa pretreatment. J Pharm Pharmacol 1991, 43, 325-330.
Wang, S. J.; Zeng, J.; Yang, B. K.; Zhong, Y. M. Bioavailability of caffeic acid in rats and its absorption properties in the Caco-2 cell model. Pharm Biol 2014, 52, 1150-1157.
Rais, R.; Thomas, A. G.; Wozniak, K.; Wu, Y.; Jaaro-Peled, H.; Sawa, A.; Strick, C. A.; Engle, S. J.; Brandon, N. J.; Rojas, C.; et al. Pharmacokinetics of oral D-serine in D-amino acid oxidase knockout mice. Drug Metab Dispos 2012, 40, 2067-2073.
Ferraris, D.; Duvall, B.; Ko, Y. S.; Thomas, A. G.; Rojas, C.; Majer, P.; Hashimoto, K.; Tsukamoto, T. Synthesis and biological evaluation of D-amino acid oxidase inhibitors. J Med Chem 2008, 51, 3357-3359.
van der Post, J. P.; de Visser, S. J.; de Kam, M. L.; Woelfler, M.; Hilt, D. C.; Vornov, J.; Burak, E. S.; Bortey, E.; Slusher, B. S.; Limsakun, T.; et al. The central nervous system effects, pharmacokinetics and safety of the NAALADase-inhibitor GPI 5693. Br J Clin Pharmacol 2005, 60, 128-136.
Vornov, J. J.; Wozniak, K. M.; Wu, Y.; Rojas, C.; Rais, R.; Slusher, B. S. Pharmacokinetics and pharmacodynamics of the glutamate carboxypeptidase II inhibitor 2-MPPA show prolonged alleviation of neuropathic pain through an indirect mechanism. J Pharmacol Exp Ther 2013, 346, 406-413.
Rais, R.; Hoover, R.; Wozniak, K.; Rudek, M. A.; Tsukamoto, T.; Alt, J.; Rojas, C.; Slusher, B. S. Reversible disulfide formation of the glutamate carboxypeptidase II inhibitor E2072 results in prolonged systemic exposures in vivo. Drug Metab Dispos 2012, 40, 2315-2323.
Jackson, P. F.; Tays, K. L.; Maclin, K. M.; Ko, Y. S.; Li, W.; Vitharana, D.; Tsukamoto, T.; Stoermer, D.; Lu, X. C.; Wozniak, K.; et al. Design and pharmacological activity of phosphinic acid based NAALADase inhibitors. J Med Chem 2001, 44, 4170-4175.
Jackson, P. F.; Cole, D. C.; Slusher, B. S.; Stetz, S. L.; Ross, L. E.; Donzanti, B. A.; Trainor, D. A. Design, synthesis, and biological activity of a potent inhibitor of the neuropeptidase N-acetylated alpha-linked acidic dipeptidase. J Med Chem 1996, 39, 619-622.
Majer, P.; Jackson, P. F.; Delahanty, G.; Grella, B. S.; Ko, Y. S.; Li, W.; Liu, Q.; Maclin, K. M.; Polakova, J.; Shaffer, K. A.; et al. Synthesis and biological evaluation of thiol-based inhibitors of glutamate carboxypeptidase II. discovery of an orally active GCP II inhibitor. J Med Chem 2003, 46, 1989-1996.
Kozikowski, A. P.; Zhang, J.; Nan, F.; Petukhov, P. A.; Grajkowska, E.; Wroblewski, J. T.; Yamamoto, T.; Bzdega, T.; Wroblewska, B.; Neale, J. H. Synthesis of urea-based inhibitors as active site probes of glutamate carboxypeptidase II: efficacy as analgesic agents. J Med Chem 2004, 47, 1729-1738.
Stoermer, D.; Liu, Q.; Hall, M. R.; Flanary, J. M.; Thomas, A. G.; Rojas, C.; Slusher, B. S.; Tsukamoto, T. Synthesis and biological evaluation of hydroxamate-Based inhibitors of glutamate carboxypeptidase II. Bioorg Med Chem Lett 2003, 13, 2097-2100.
Parellada, J.; Suarez, G.; Guinea, M. Inhibition of zinc metallopeptidases by flavonoids and related phenolic compounds: structure-activity relationships. J Enzyme Inhib 1998, 13, 347-359.
Rahn, K. A.; Slusher, B. S.; Kaplin, A. I. Glutamate in CNS neurodegeneration and cognition and its regulation by GCPII inhibition. Curr Med Chem 2012, 19, 1335-1345.
Slusher, B. S.; Vornov, J. J.; Thomas, A. G.; Hurn, P. D.; Harukuni, I.; Bhardwaj, A.; Traystman, R. J.; Robinson, M. B.; Britton, P.; Lu, X. C.; et al. Selective inhibition of NAALADase, which converts NAAG to glutamate, reduces ischemic brain injury. Nat Med 1999, 5, 1396-1402.
Yamamoto, T.; Nozaki-Taguchi, N.; Sakashita, Y.; Inagaki, T. Inhibition of spinal N-acetylated-alpha-linked acidic dipeptidase produces an antinociceptive effect in the rat formalin test. Neuroscience 2001a, 102, 473-479.
Yamamoto, T.; Nozaki-Taguchi, N.; Sakashita, Y. Spinal N-acetyl-alpha-linked acidic dipeptidase (NAALADase) inhibition attenuates mechanical allodynia induced by paw carrageenan injection in the rat. Brain Res 2001b, 909, 138-144.
Chen, S. R.; Wozniak, K. M.; Slusher, B. S.; Pan, H. L. Effect of 2-(phosphono-methyl)-pentanedioic acid on allodynia and afferent ectopic discharges in a rat model of neuropathic pain. J Pharmacol Exp Ther 2002, 300, 662-667.
Carpenter, K. J.; Sen, S.; Matthews, E. A.; Flatters, S. L.; Wozniak, K. M.; Slusher, B. S.; Dickenson, A. H. Effects of GCP-II inhibition on responses of dorsal horn neurones after inflammation and neuropathy: an electrophysiological study in the rat. Neuropeptides 2003, 37, 298-306.
Ghadge, G. D.; Slusher, B. S.; Bodner, A.; Canto, M. D.; Wozniak, K.; Thomas, A. G.; Rojas, C.; Tsukamoto, T.; Majer, P.; Miller, R. J.; et al. Glutamate carboxypeptidase II inhibition protects motor neurons from death in familial amyotrophic lateral sclerosis models. Proc Natl Acad Sci USA 2003, 100, 9554-9559.
Ghose, S.; Chin, R.; Gallegos, A.; Roberts, R.; Coyle, J.; Tamminga, C. Localization of NAAG-related gene expression deficits to the anterior hippocampus in schizophrenia. Schizophr Res 2009, 111, 131-137.
Vornov, J. J.; Peters, D.; Nedelcovych, M.; Hollinger, K.; Rais, R.; Slusher, B. S. Looking for Drugs in All the Wrong Places: Use of GCPII Inhibitors Outside the Brain. Neurochem Res 2020, 45, 1256-1267.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 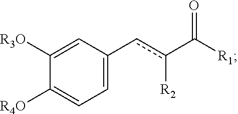
wherein: [symbol]
indicates that the bond can be a single or a double bond;
R₁ is:
—OR₈, wherein R₅ is H or C₁-C₈ alkyl; or
—NR₇R₈, wherein R₇ H or C₁-C₄ alkyl and R₈ is selected from the group consisting of C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₈ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8;
provided that if R₁ is —OR₅, then R₃, R₄, and R₅ cannot all be H, and that if R₃ and R₄ are each H, then R₇ and R₈ cannot both be methyl; and
stereoisomers and pharmaceutically acceptable salts thereof.

2. The compound of claim 1, wherein R₁ is —OR₅, and R₅ is selected from the group consisting of methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

4. The compound of claim 1, wherein R₁ is —NR₇R₈, and R₇ is H or C₁-C₄ alkyl and R₈ is selected from the group consisting of C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

5. The compound of claim 1, wherein R₂ is —NR₁₁R₁₂, wherein Ru is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₈ and R₁₆ are each H or C₁-C₄ alkyl, and p is an integer selected from the group consisting of 0, 1, 2, 3, 4, 5, 6, 7, and 8.

6. The compound of claim 1, wherein R₃ and R₄ are each H, provided that if R₁ is —OR₅, then R₅ cannot be H.

7. The compound of claim 1, wherein R₃ and R₄ are each independently selected from the group consisting of —C(═O)—CH₃, —C(═O)—C(CH₃)₃, and —CH₂—O—C(═O)—O—CH(CH₃)₂.

8. The compound of claim 1, wherein the compound of formula (I) is selected from the group consisting of:

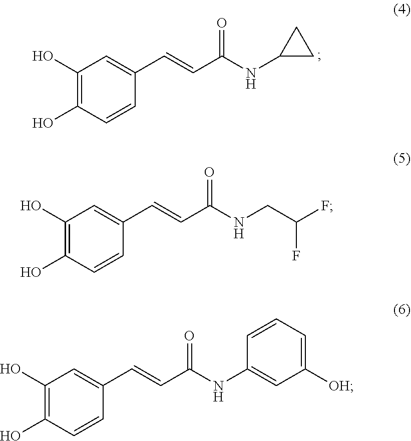 [SMILES: O=C(/C=C/c1ccc(O)c(O)c1)NC1CC1 ; ";" ; O=C(/C=C/c1ccc(O)c(O)c1)NCC(F)F ; O=C(/C=C/c1ccc(O)c(O)c1)Nc1cccc(O)c1]

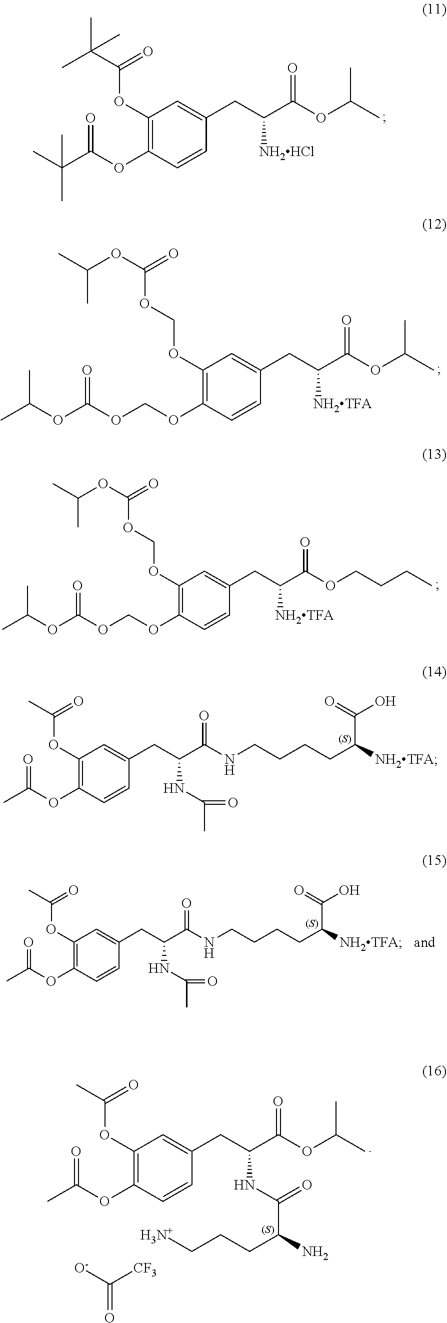

9. A pharmaceutical composition comprising a compound of claim 1 and a pharmaceutically acceptable carrier, diluent, or excipient.

10. A method for treating a disease, disorder, or condition associated with excess glutamate carboxypeptidase II (GCPII), the method comprising administering to a subject in need of treatment thereof, a therapeutically effective amount of L-DOPA, caffeic acid, D-DOPA, or a compound of formula (I): 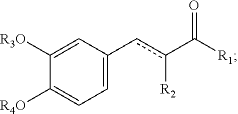
wherein: [symbol]
indicates that the bond can be a single or a double bond;
R₁ is:
—OR₅, wherein R₅ is selected from the group consisting of H, C₁-C₈ alkyl, and —O—(CH₂)ₙ—R₆, wherein n is an integer selected from the group consisting of 0, 1, 2, 3, 4, 5, 6, 7, and 8 and R₆ is substituted or unsubstituted aryl or heteroaryl; or
—NR₇R₈, wherein R₇ and R₈ are each independently selected from the group consisting of H, C₁-C₄ alkyl, C₃-C₆ cycloalkyl, C₁-C₈ alkoxyl, unsubstituted or substituted aryl or heteroaryl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8;
R₂ is H or —NR₁₁R₁₂, wherein R₁₁ and R₁₂ are each independently selected from the group consisting of H, C₁-C₄ alkyl, and —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8;
R₃ and R₄ are each independently H or —C(═O)—R₁₇, wherein R₁₇ is C₁-C₈ alkyl or —(CH₂)ₜ—O—C(═O)—O—R₁₈, wherein R₁₈ is C₁-C₈ alkyl, and t is an integer selected from the group consisting of 0, 1, 2, 3, 4, 5, 6, 7, and 8; and
stereoisomers and pharmaceutically acceptable salts thereof.

11. The method of claim 10, wherein R₁ is —OR₅, and R₈ is selected from the group consisting of H, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, and n-octyl.

12. The method of claim 10, wherein R₁ is —OR₅, and R₈ is H or —O—(CH₂)ₙ—R₆, wherein R₆ is substituted or unsubstituted phenyl.

13. The method of claim 10, wherein R₁ is —NR₇R₈, and R₇ is H or C₁-C₄ alkyl and R₈ is selected from the group consisting of H, C₁-C₄ alkyl, C₃-C₆ cycloalkyl, unsubstituted or substituted phenyl, —(CH₂)ₘ—R₉, wherein R₉ is —OR₁₀ or CHX₂, wherein R₁₀ is H or C₁-C₄ alkyl, and each X is halogen, and —(CH₂)ₘ—CH(NH₂)(COOH), wherein each m is independently an integer selected from the group consisting of 0, 1, 2, 3, 4, 5, 6, 7, and 8.

14. The method of claim 10, wherein R₂ is —NR₁₁R₁₂, wherein Ru is H and R₁₂ is H or —C(═O)—R₁₃, wherein R₁₃ is C₁-C₄ alkyl or —C(NH₂)—(CH₂)ₚ—R₁₄, wherein R₁₄ is C₁-C₄ alkyl or —NR₁₅R₁₆, wherein R₁₅ and R₁₆ are each H or C₁-C₄ alkyl, and p is an integer selected from the group consisting of 0, 1, 2, 3, 4, 5, 6, 7, and 8.

15. The method of claim 10, wherein R₃ and R₄ are each H.

16. The method of claim 10, wherein R₃ and R₄ are each independently selected from the group consisting of —C(═O)—CH₃, —C(═O)—C(CH₃)₃, and —CH₂—O—C(═O)—O—CH(CH₃)₂.

17. The method of claim 10, wherein the compound of formula (I) is selected from the group consisting of:

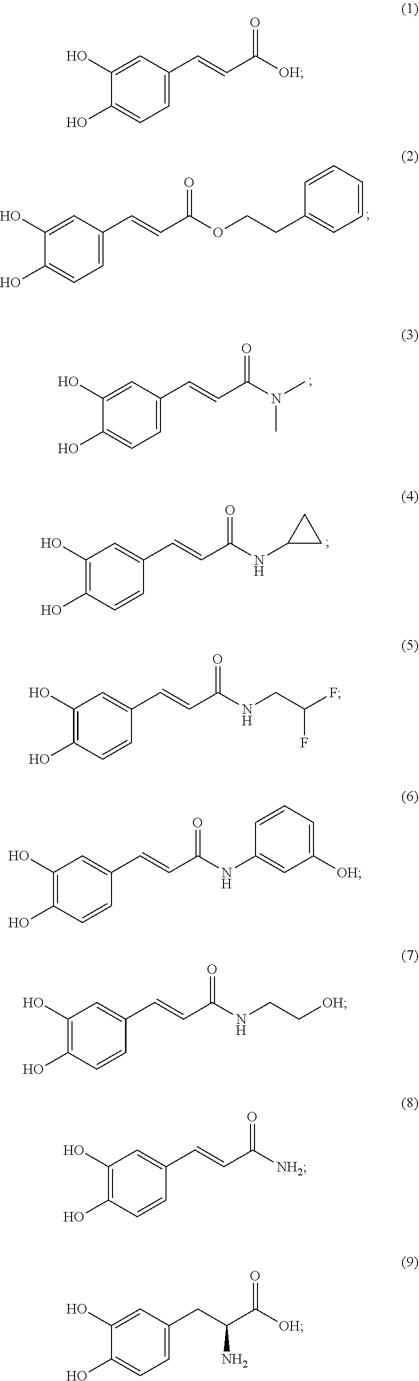

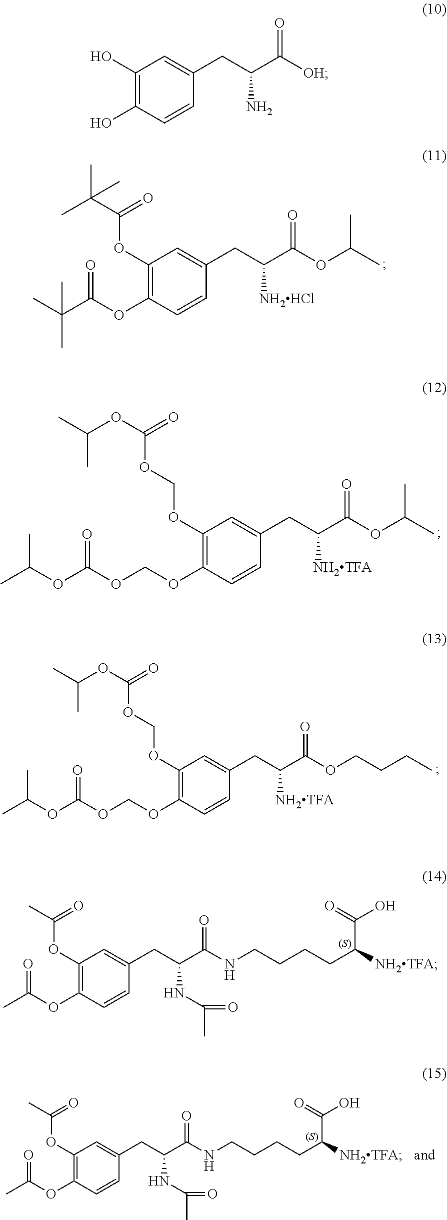

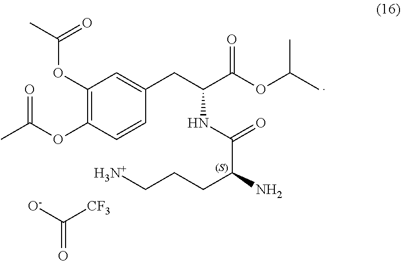

18. The method of claim 10, wherein the disease, disorder, or condition is a central nervous system (CNS) or an inflammatory disease, disorder, or condition.

19. The method of claim 10, wherein the disease, disorder, or condition is selected from the group consisting of ischemic stroke, traumatic brain injury, traumatic spinal cord injury, amyotrophic lateral sclerosis (ALS), Parkinson's disease, Huntington's disease, epilepsy, neuropathic pain, inflammatory pain, chemotherapy-induced neuropathy, diabetic neuropathy, schizophrenia, multiple sclerosis, cognition impairment, brain cancer, HIV-associated neurocognitive disorder, cognition impairment associated with neurodegenerative or neuropsychiatric conditions, inflammatory bowel disease (IBD), and neurological conditions as a result of drug abuse or drug addiction.

20. The method of claim 10, comprising intranasally administering a therapeutically effective amount of a compound of formula (I).

21. The method claim 1, further comprising administering L-DOPA, caffeic acid, D-DOPA, or a compound of formula (I) in combination with a D-amino acid oxidase (DAAO) inhibitor.

22. The method of claim 21, wherein the DAAO inhibitor is selected from sodium benzoate, risperidone, blonanserin, and luvadaxistate (TAK-831).

23. The method of claim 22, wherein the DAAO inhibitor comprises sodium benzoate.

24. The method of claim 1, further comprising administering L-DOPA in combination with carbidopa.
